(19) United States
(12) Patent Application Publication
(10) Pub. No.: US 2014/0378528 A1
(43) Pub. Date: Dec. 25, 2014

(54) BIOMARKERS OF MIR-34 ACTIVITY

(71) Applicant: Mirna, Austin, TX (US)

(72) Inventors: Andreas Bader, Austin, TX (US); Jane Zhao, Austin, TX (US)

(21) Appl. No.: 14/313,471

(22) Filed: Jun. 24, 2014

Related U.S. Application Data
(60) Provisional application No. 61/870,997, filed on Aug. 28, 2013.
(60) Provisional application No. 61/838,847, filed on Jun. 24, 2013.

(51) Int. Cl. C12N 15/113; A61K 31/7105; A61K 45/06; C12Q 1/68; G01N 33/574

(52) U.S. Cl. CPC C12N 15/113; C12Q 1/6886; G01N 33/5748; G01N 33/57496; A61K 45/06; A61K 31/7105; C12N 2310/141; C12N 2320/30; C12Q 2600/158; C12Q 2600/106
USPC 514/44 A; 435/6.11; 435/6.12; 435/7.92; 435/7.1; 506/9; 435/7.4

(57) ABSTRACT

This invention is based in part on the discovery that miR-34 is independent of p53. It has been discovered that miR-34 functions in a TP53-independent tumor suppression pathway. Specifically, miR-34-induced inhibition of cancer cell growth was found to be the same in p53-normal and p53-deficient cells. Thus, miR-34 has a more central role during tumor suppression that is uncoupled from p53. In the absence of p53, miR-34, unlike certain other miRNAs, is sufficient to induce an up-regulation of genes known to be regulated by p53, including but not limited to p21^{CIP1/WAF1} (CDKN1A), PUMA, BAX, NOXA, PHLDA3, and MDM2 and a down-regulation of HDAC1. Therefore, these biomarkers can be used as biomarkers of miR-34 activity. The invention is further based on the discovery that some of these biomarkers are indispensable for a therapeutic response to miR-34 activity, and are thus prerequisite biomarkers of miR-34 activity.

### CROSS REFERENCE TO RELATED APPLICATIONS

This application claims benefit of priority to U.S. Ser. No. 61/838,847 filed Jun. 24, 2013 and U.S. Ser. No. 61/870,997 filed Aug. 28, 2013, the contents of which are incorporated herein by reference in their entirety.

### US GOVERNMENT RIGHTS

This invention was made in the performance of work under National Institute of Health grant Nos. 1R43CA134071 and 1R43CA137939. The US government may have rights in this invention.

### SEQUENCE LISTING

The instant application contains a Sequence Listing which has been submitted in ASCII format via EFS-Web and is hereby incorporated by reference in its entirety. Said ASCII copy, created on June 23, is named “112172-241 Sequence Listing TEXT” and is 30,360 bytes.

### FIELD OF THE INVENTION

The present invention relates generally to cancer therapy, and more particularly, it concerns methods and compositions involving microRNA (miRNAs) molecules, such as miR-34, in disease treatment, diagnosis, prognosis, and/or evaluation of disease progression.

### BACKGROUND

MicroRNA-34 (miR-34) is a potent tumor suppressor that shows a loss of function in many solid and hematological cancer types (Lodygin et al., Cell Cycle 7(16):2591-600 (2008); Gallardo et al., Carcinogenesis 30(11):1903-9 (2009); Chim et al., Carcinogenesis 31:745-750 (2010)). It inhibits a broad range of cancer cells, presumably by repressing a plethora of oncogenes that control proliferation, senescence, apoptosis and metastasis (Hermeking, Cell Death Differ 17(2):193-9 (2010); Bader, Front Genet 3(120) (2012)). miR-34 can also interfere with the growth of cancer stem cells (Ji et al., PLoS One, 4(8):e6816 (2009); Liu et al., Nat Med 17(2):211-5 (2011)), providing a strong rationale for the development of a miR-34 therapy. Evidence for the therapeutic application of miR-34 has been generated in murine tumor models of lung, liver, prostate and lymphoma that showed robust tumor inhibition in response to the systemic delivery of nanoparticles loaded with synthetic miR-34 mimics (Bader, Front Genet 3(120) (2012); Liu et al., Nat Med 17(2):211-5 (2011); Trang et al., Mol Ther 19(6):1116-22 (2011); Wiggins et al., Cancer Res 70(14):5923-30 (2010); Craig et al., Leukemia (2012)). A miR-34-based therapy is currently in Phase I clinical trials.

Much insight into the role of miR-34 has been added by recent reports demonstrating that the tumor suppressor TP53 (p53) transcriptionally induces the expression of all three miR-34 family members, miR-34a/b/c (Bommer et al., Curr Biol, 17(15):1298-307 (2007); Chang et al., Mol Cell 26(5):745-52 (2007); He et al., Nature 447(7148):1130-4 (2007); Raver-Shapira et al., Mol Cell 26(5):731-43 (2007); Tarasov et al., Cell Cycle 6(13):1586-93 (2007)). TP53 also elevates the endogenous levels of miR-215, miR-192 and miR-194, all of which have the ability to inhibit cancer cell growth in culture (Braun et al., Cancer Res 68(24):10094-104 (2008); Georges et al., Cancer Res 68(24):10105-12 (2008); Pichiorri et al., Cancer Cell 18(4):367-81 (2011)). Although miR-215 and miR-192 are encoded on separate genomic loci, they share identical seed sequences (90.5% overall sequence homology) and thus may be collectively referred to as miR-215/192. For some miRNAs, the positive regulation between TP53 and miRNA is reciprocal miR-215/192 stimulates TP53 activity by repressing MDM2 (also referred to as HDM2), a ubiquitin ligase that negatively regulates TP53 stability via proteasomal degradation (Pichiorri et al., Cancer Cell 18(4):367-81 (2011); Momand et al., Cell 69(7):1237-45 (1992); Oliner et al., Nature 358(6831):80-3 (1992)). Similarly to miR-215/192, miR-34a functions in a positive feedback loop to TP53 by repressing SIRT1 (silent information regulator 1), a NAD-dependent deacetylase that deactivates TP53, MDM4, encoding a MDM2-like protein that negatively regulates TP53 transactivation, and YY1, encoding a transcription factor binds to a subset of TP53 DNA binding sites (Yamakuchi et al., Proc Natl Acad Sci USA 105(36):13421-6 (2008); Mandke et al., PLoS One 7(8):e42034 (2012)). Therefore, it is possible that TP53 is a functional requirement for the miR-34-induced phenotype.

Given the high mutation rate of TP53 in cancer, this prerequisite would substantially limit the application of a miR-34-based therapy to patients with intact TP53. While available data support the view that TP53 enhances the inhibitory activity of miR-215/192 (Pichiorri et al., Cancer Cell 18(4):367-81 (2011)), a requirement for TP53 in miR-34-induced tumor suppression is controversial, and an actual contribution of TP53 is unknown.

Therefore, although significant advances have been made in the use of microRNAs in the diagnosis and treatment of various cancers, particular microRNA therapies may be not effective or less effective in certain subpopulations. There is a need for better solutions in certain subpopulations, for example, in TP53-deficient cells or cancer types. The ability to predict whether a patient would be responsive to a particular therapy or to quickly determine whether the patient is responding to a particular therapy could reduce the time and cost associated with cancer treatment, while providing better outcomes.

### SUMMARY

This invention is based at least in part on the discovery that functions of miR-34 are independent of p53. It has been discovered that miR-34 functions in a TP53-independent tumor suppression pathway. Specifically, miR-34-induced inhibition of cancer cell growth was found to be the same in p53-normal and p53-deficient cells. Thus, miR-34 has a more central role during tumor suppression that is uncoupled from p53. In the absence of p53, miR-34, unlike certain other miRNAs, is sufficient to induce an up-regulation of genes known to be regulated by p53, including but not limited to p21^{WAF1/CIP1} (CDKN1A), PUMA, BAX, NOXA, PHLDA3, and MDM2 and a down-regulation of HDAC1. Therefore, the invention provides that these biomarkers can be used as biomarkers of miR-34 activity. In various embodiments, the prerequisite biomarker(s) is a DNA, mRNA, or protein (or a combination thereof). Where a prerequisite biomarker is DNA, it can be DNA of a gene that is not silenced and that is free of any inactivating mutation(s). The invention is further based on the discovery that certain of these biomarkers are indispensable for a therapeutic response to miR-34 activity, and are thus prerequisite biomarkers of miR-34 activity.

### Methods of Treatment

In some embodiments, methods of treating a subject having cancer are provided. Two general methods of treatment are provided. The first set of methods includes a screening step that employs one or more prerequisite biomarkers of miR-34 activity to determine whether a miR-34 therapy is the appropriate method of treatment for a subject. The second set of methods includes measuring relative levels of biomarkers of miR-34 activity in a subject to determine the subject's response to treatment by a miR-34 therapeutic.

The first set, in some embodiments, provides a method of treating a subject having cancer that includes:

(a) screening the subject for the presence or absence of at least one prerequisite biomarker of miR-34 activity;

(b) administering a miR-34 therapeutic to the subject if the prerequisite biomarker(s) for miR-34 activity is determined to be present; and

(c) administering an alternative therapy to the subject if the prerequisite biomarker(s) for miR-34 activity is absent,

thereby treating the subject. The at least one prerequisite biomarker can be selected from the group consisting of p21^{CIP1/WAF1} PUMA, BAX, NOXA, PHLDA3, MDM2, and HDAC1. In some aspects, the at least one prerequisite biomarker comprises p21^{CIP1/WAF1} and in some aspects, the sole prerequisite biomarker for which the subject is screened is p21^{CIP1/WAF1}. For example, the at least one prerequisite biomarker can selected from the group consisting of p21CIP1WAF1, PUMA, BAX, NOXA, PHLDA3, and MDM2 and presence of the at least one prerequisite biomarker comprises an expression level or activity level that is equal to or above a reference level. In another example, the at least one prerequisite biomarker is HDAC1 and presence of the at least one prerequisite biomarker comprises an expression level or activity level that is equal to or below a reference level.

The second set, in some embodiments, provides a method of determining a response to a cancer therapy in a subject being treated with a miR-34 therapeutic, said method includes:

(a) measuring a first level of at least one biomarker of miR-34 activity in the subject;

(b) administering a miR-34 therapeutic to the subject;

(c) measuring a second level of the biomarker(s) in the subject;

(d) further administering a miR-34 therapeutic to the subject if the second level relative to the first level indicates efficacy of the miR-34 therapeutic; and

(e) administering an alternative therapy to the subject if the second level relative to the first level indicates insufficient efficacy of the miR-34 therapeutic,

thereby determining the response of the subject to cancer therapy and providing an appropriate treatment. The biomarker can be selected from the group consisting of p21^{CIP1/WAF1}, p53, PUMA, BAX, NOXA, PHLDA3, MDM2, and HDAC1. In some aspects, the miR-34 therapeutics in steps (b) and (d) are the same.

The subject can have lung cancer, pancreatic cancer, cancer in the liver, hepatocellular carcinoma, breast cancer, colorectal cancer, head and neck cancer, prostate cancer, brain cancer, stomach cancer, bladder cancer, esophageal cancer, or colon cancer. In some aspects, the subject has been determined to have p21^{CIP1/WAF1}-positive cancer cells. For example, the subject can be determined to have p21^{CIP1/WAF1}-positive cancer cells and p53-deficient cancer cells. For example, the subject can be determined to have cancer cells having a p21^{CIP1/WAF1} wild type (+/+) or heterozygous (+/−) gene and a homozygously inactivated (−/−) p53 gene. In another example, the subject can be determined to have cancer cells having a p21^{CIP1/WAF1} wild type (+/+) or heterozygous (+/−) gene and lacking functional p53 protein. In another example, the subject can be determined to have functional p21^{CIP1/WAF1} protein and non-functional or missing p53 protein. The subject can have a hematological malignancy, for example, a leukemia (acute lymphoblastic leukemia (ALL), acute myelogenous leukemia (AML), acute lymphocytic leukemia, chronic lymphocytic leukemia (CLL), small lymphocytic lymphoma (SLL), chronic myelogenous leukemia (CML), acute monocytic leukemia (AMoL), or other leukemias); a lymphoma (Hodgkin's lymphomas (all four subtypes), follicular lymphoma, B-cell lymphoma, or non-Hodgkin's lymphomas (all subtypes)); a myeloma (e.g., multiple myeloma); or a myelodystplastic syndrome.

The miR-34 therapeutic can include miR-34 and can further optionally include at least one of miR-215 and miR-192. miR-34 can be miR-34a, b, or c, or miR-449a, b, or c. A level of cellular proliferation can decrease after administration of the miR-34 therapeutic. A tumor size or progression can decrease or slow after administration of the miR-34 therapeutic.

In some aspects, the alternative therapy can be a non-miR-34 microRNA therapy or a non-microRNA therapy. For example, the alternative therapy can be selected from discontinued therapy, chemotherapy, radiotherapy, surgery, palliative therapy, miR-215 and miR-192. The alternative therapy can be any combination of non-miR-34 microRNA therapies and non-microRNA therapies. For example, the alternative therapy can include at least one of miR-215 and miR-192.

### BRIEF DESCRIPTION OF THE FIGURES

FIG. 1 shows common and separate tumor suppression pathways of TP53 and miR-34.

FIGS. 2A-2E show endogenous mRNA and miRNA levels after induction of TP53 genes in isogenic cancer cell lines. mRNA levels of TP53 are normalized to those in TP53^{+/-} cells (SW48, HCT116, RKO) or TP53^{-/-} cells (MCF10A; expression=1). Data from DLD-1 cells are normalized to DLD-1^{-/SIL} cells. All other data are normalized to expression levels in TP53-deficient cells (−/−; relative expression=1). Averages and standard deviations are shown. n, not detected; *, data are normalized to a standardized PCR threshold due to absence in reference cells.

FIGS. 3A-3D show the inhibition of cancer cell proliferation by miR-34a in isogenic cancer cell lines. Data are normalized to mock-transfected cells. Averages, standard deviations and non-linear regression trendlines are shown.

FIGS. 4A-4D show the inhibition of cancer cell proliferation by miR-34c in isogenic cancer cell lines. Data are normalized to mock-transfected cells. Averages, standard deviations and non-linear regression trendlines are shown.

FIGS. 5A-5D show the inhibition of cancer cell proliferation by miR-215 in isogenic cancer cell lines. Data are normalized to mock-transfected cells. Averages, standard deviations and non-linear regression trendlines are shown.

FIGS. 6A-6D show the inhibition of cancer cell proliferation by miR-192 in isogenic cancer cell lines. Data are normalized to mock-transfected cells. Averages, standard deviations and non-linear regression trendlines are shown.

FIG. 7A shows the inhibition of cancer cell proliferation by miR-34a in isogenic RKO cells. Cellular proliferation was determined by AlamarBlue®. Averages and standard deviations are shown.

FIG. 7B shows the inhibition of cancer cell proliferation by miR-34c in isogenic RKO cells. Cellular proliferation was determined by AlamarBlue®. Averages and standard deviations are shown.

FIGS. 8A and 8B show endogenous expression levels of target genes functioning in the miR-34a/TP53 axis in isogenic SW48 cells transfected with either miR-34a or miR-215. FIG. 8A shows the levels for SIRT1, MDM4, BCL2, MET, and p53 and FIG. 8B shows the levels for PUMA, p21, and MDM2. Values are normalized to those in mock-transfected cells (=1). Averages and standard deviations of duplicate experiments are shown. n, not detected.

FIGS. 9A and 9B show endogenous levels of p21^{CIP1/WAF1}, PUMA, BAX, NOXA, PHLDA3, MDM2 mRNA in isogenic cell lines transfected with miR-34a. All values are normalized to those in mock-transfected cells (=1). Averages are shown.

FIG. 10 shows the miR-34a binding site in the 3′UTR of the HDAC1 transcript. Base pairing of miR-34a with wild-type (wt) and mutated (mut) HDAC1 3′UTR sequences is shown. Lower case, miR-34a residues; upper case, mRNA residues; highlighted bases presumably involved in base pairing; bold, miRNA seed sequence; underlined, mutated residues.

FIG. 11 shows HDAC1 mRNA levels in isogenic SW48 cells transfected with miR-34a. Values are normalized to those in mock-transfected cells.

FIG. 12A shows expression of a luciferase transcript fused to the HDAC1 3′UTR in SW48 colorectal cancer cells and H1299 lung cancer cells transfected with miR-34a or miR-215. Relative light units were normalized to those in miR-215-transfected cells (100%). P values were derived from two-tailed Student's t-tests. n.s., not statistically significant.

FIG. 12B shows the correlation between HDAC1 mRNA and miR-34 levels in a set of 14 tumors from NSCLC patients normalized to the respective normal adjacent tissue. Endogenous expression levels were determined by qRT-PCR. Correlation coefficient was generated by the Pearson's method; the P value was calculated by F test (Graphpad).

FIG. 13 shows the effect of transfection with miRNAs and siRNAs on biomarker protein expression in isogenic TP53-negative SW48 cells.

FIG. 14 shows the effect of transfection with miRNAs and siRNAs on biomarker protein expression in isogenic TP53-negative RKO cells.

FIG. 15 shows the effect of transfection with miRNAs and siRNAs on biomarker protein expression in isogenic TP53-positive RKO cells.

FIGS. 16A-16G show endogenous mRNA expression levels of various mRNAs in isogenic SW48 cells transiently transfected with siRNAs. Expression levels are normalized to those of cells transfected with negative control siRNA (si-NC). Averages and standard deviations of triplicate experiments are shown.

FIG. 17 shows cellular proliferation of isogenic SW48 cells transfected with siRNAs and miRNAs. Values are normalized to mock transfections (=100%). Averages and standard deviations are shown. P values are derived from two-tailed Student's t-tests. The dotted line denotes the level of cellular proliferation in cells transfected with miR-34a.

FIG. 18A shows the effect of trichostatin A on proliferation in isogenic SW48 cell lines. Values are normalized to non-treated cells (NT).

FIG. 18B shows a qRT-PCR analysis measuring p21^{CIP1/WAF1} mRNA levels using RNA samples from cells treated with trichostatin A. Values are normalized to non-treated cells (NT).

FIG. 19 shows a non-linear regression analysis of p21 mRNA expression levels and proliferation rates in isogenic RKO cells transfected with increasing concentrations of miR-34a. All values are normalized to those in mock-transfected cells (=1). Averages are shown. Standard deviations are included but are too small to be visible in the graph.

FIG. 20 shows biomarker protein expression measured by Western analysis after miRNA mimics and siRNA were transiently transfected into isogenic RKO cells.

FIG. 21 shows cell proliferation in isogenic RKO cells after transient transfection with miRNA mimics and siRNA. Proliferation data was assessed by AlamarBlue®. Values are normalized to cells transfected with negative control (100%). Averages and standard deviations are shown. P values were derived from a 2-tailed Student's t-test.

FIG. 22 shows cell proliferation in isogenic SW48 cells after transient transfection with miRNA mimics and siRNA. Proliferation data was assessed by AlamarBlue® and are normalized to cells transfected with negative control (100%). Averages and standard deviations are shown. P values were derived from a 2-tailed Student's t-test.

FIG. 23 shows cell proliferation in isogenic Hep3B hepatocellular carcinoma cells after transient transfection with miRNA mimics and siRNA. Proliferation data was assessed by AlamarBlue® and are normalized to cells transfected with negative control (miNC+siNC; 100%). Averages and standard deviations are shown. An siRNA against EG5 (Kifl1) served as a positive control for inhibition of cancer cell growth.

FIG. 24A shows upregulation of p21^{CIP1/WAF1} in a mouse model of liver cancer treated with a miR-34a-based therapy. Mice carrying human Hep3B tumors orthotopically grown in liver were given a single dose of the miR-34a-based therapy via intravenous tail vein injection (n=3). As controls, tumor-bearing mice were injected with either empty liposomes (n=2) or liposomes loaded with a negative control miRNA (miR-NC2; n=2). After 24 hours, tumor tissues were collected and protein lysates were probed by Western analysis. Actin expression served as a loading control.

FIG. 24B shows upregulation of p21^{CIP1/WAF1} in cultured Hep3B liver cancer cells. Cells were transiently transfected with either miR-34a or miR-NC2. Protein lysates were probed for p21^{CIP1/WAF1} expression by Western analysis. Actin was used as a loading control.

### DETAILED DESCRIPTION

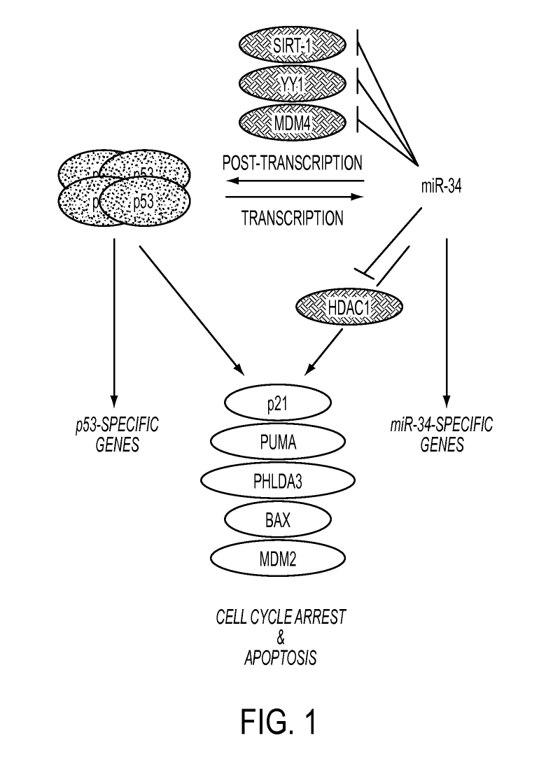

This invention is based at least in part on the discovery that functions of miR-34 are independent of p53. It has been discovered that miR-34 functions in a TP53-independent tumor suppression pathway. Specifically, miR-34-induced inhibition of cancer cell growth was found to be the same in p53-normal and p53-deficient cells. Thus, miR-34 has a more central role during tumor suppression that is uncoupled from p53. In the absence of p53, miR-34, unlike certain other miRNAs, is sufficient to induce an up-regulation of genes known to be regulated by p53, including but not limited to p21^{CIP1/WAF1} (CDKN1A), PUMA, BAX, NOXA, PHLDA3, and MDM2 and a down-regulation of HDAC1. Therefore, these biomarkers can be used as biomarkers of miR-34 activity. The invention is further based on the discovery that some of these biomarkers are indispensable for a therapeutic response to miR-34 activity, and are thus prerequisite biomarkers for miR-34 activity. It was also found that p21^{CIP1/WAF1} is a critical effector molecule downstream of miR-34. In contrast to p21^{CIP1/WAF1}, p53 can be bypassed by miR-34 to function as a p53-independent tumor suppressor. FIG. 1 illustrates the common and separate suppression pathways of miR-34 and p53.

miR-34 replacement has emerged as a promising approach to treat cancer. miR-34 is transcriptionally induced by p53. However, miR-34 also activates p53 in a positive feedback loop which was suspected to be required for the miR-34 phenotype. The functional relationships between p53 and miR-34, and that of other p53-regulated miRNAs, including miR-215/192, has been determined using a panel of isogenic cancer cell lines that differ only with respect to their endogenous p53 status. miR-34-induced inhibition of cancer cell growth is the same in p53-normal and p53-deficient cells. In contrast to miR-34, miR-215/192 functions through p53. In the absence of p53, miR-34, but not miR-215/192, is sufficient to induce an up-regulation of the cell cycle-dependent kinase inhibitor p21^{CIP1/WAF1} (CDKN1A). As such, miR-34 can be therapeutically active in patients irrespective of their p53 status since, unlike other downstream targets of p53, miR-34 exhibits similar activity in p53-normal and p53-deficient cells. The p53-independent functions of miR-34 also have important therapeutic implications and can help predict which patients are most likely to respond to a particular therapy, as well as determine how well a patient is responding to a particular therapy.

Histone deacetylase 1 (HDAC1) is identified as a direct target of miR-34, and repression of HDAC1 leads to an induction of p21^{CIP1/WAF1} and mimics the miR-34 cellular phenotype. Depletion of p21^{CIP1/WAF1} specifically interferes with the ability of miR-34 to inhibit cancer cell proliferation. The data suggest that miR-34 controls a tumor suppressor pathway previously reserved for p53 and provides an attractive therapeutic strategy for cancer patients irrespective of their p53 status.

The established paradigm views miR-34 as a cellular effector molecule that functions downstream of TP53 by repressing genes involved in cell cycle progression and apoptosis. Our data, however, suggest that miR-34 has a more central role that is independent of and in parallel to p53. Support for this thesis is provided here. The existence of a separate miR-34 pathway is further corroborated by the TP53-independent transcriptional regulation of the miR-34a gene (Christofferson et al., Cell Death Differ 17(2):236-45 (2009)), as well as observations made in miR-34 knock-out mice that show an intact p53 response in the absence of miR-34 (Concepcion et al., PLoS Genet 8(7):e1002797 (2012)). miR-34 and p53 may create an interface of two pathways with overlapping functions and activate each other reciprocally—p53 via transcription, and miR-34 via post-transcriptional repression of SIRT1, YY1 and MDM4.

In p53-deficient cells, the miR-34-induced expression of p21^{CIP1/WAF1} is an indirect effect of HDAC1 repression. HDAC1 has previously been implicated in the regulation of the p21 gene (CDKN1A). Supporting evidence comes from HDAC1-deficient embryonic stem cells that show elevated levels of p21^{CIP1/WAF1} and p53-mutated human osteosarcoma cells in which p21^{CIP1/WAF1} expression was induced after treatment with the HDAC inhibitor Trichostatin A (TSA) (Sowa et al., Biochem Biophys Res Commun (1997); Lagger et al., Embo J 21(11):2672-81 (2002)). In these studies, two Sp1 binding sites in the CDKN1A promoter were identified as TSA-responsive elements, suggesting that in the absence of TP53 Sp1 is the transcription factor that controls the activation of the CDKN1A gene. Our data indicate that p21^{CIP1/WAF1} is a functional prerequisite for miR-34a function. However, the effects of p21^{CIP1/WAF1} depletion varied between cell lines. p21^{CIP1/WAF1} depletion completely abolished the anti-proliferative activity of miR-34a in RKO cells and merely weakened it in SW48 cells. Likewise, the inhibitory activity of miR-34a was not fully reduced but significantly lessened in TP53-negative Hep3B hepatocarcinoma cells that lack p21^{CIP1/WAF1} (data not shown). In contrast, previous studies did not reveal a p21^{CIP1/WAF1}-dependent miR-34a phenotype in HCT116^{p21-/-} cells (He et al., Nature 447(7148):1130-4 (2007)). Therefore, the effects of p21^{CIP1/WAF1} depletion appear to vary across cell lines. It is possible that the miR-34 phenotype is additionally controlled by other molecular events that are subject to change in cancer. Although deletion of CDKN1A can lead to spontaneous tumor formation in mice, somatic loss-of-function mutations in human cancer are rare (Lagger et al., Embo J 21(11):2672-81 (2002)). However, reduced expression has been noted in colorectal, cervical, esophageal and lung cancers, and in some of these, this is due to the hypermethylation of the CDKN1A promoter (Dotsch et al., Cold Spring Harb Perspect Biol 2(9):a004887 (2010); Toledo et al., Cancer Cell 9(4):273-85 (2006)). Thus, a miR-34 mimic may be less active in cancers with silenced or reduced CDKN1A, which should be considered as a “predictive” marker of a response to a miR-34 therapy, i.e., a prerequisite marker for miR-34 activity.

The miR-34-specific induction of p21^{CIP1/WAF1} offers an explanation for its invariable ability to inhibit p53-normal and p53-deficient cells. This is in stark contrast to miR-215/192 that is unable to induce p21^{CIP1/WAF1} in the absence of p53 and, consequently, has reduced inhibitory activity in TP53^{-/-} cells. The data generated with miR-215/192 fit a model described previously in which miR-215/192 functions in a positive feedback loop to TP53 via repression of MDM2 (Pichiorri et al., Cancer Cell 18(4):367-81 (2011)). Interestingly, the reported positive feedback from miR-34 to TP53 via SIRT1, YY1 or MDM4 does not seem contribute to the anti-proliferative miR-34 phenotype despite the fact that these proteins were expressed and downregulated by miR-34a. Given the modest effects of siRNAs against these gene products, it is possible that they do not participate in an immediate anti-proliferative miR-34 response but may reveal added effects at later time points.

miR-34a-induced inhibition of cancer cell proliferation is independent of p53 and suggests that a miR-34 therapy is effective in cancer patients irrespective of p53 status. The ability of miR-34 to repress HDAC1 and to induce p21^{CIP1/WAF1} significantly strengthens its position as a central tumor suppressor and complements its function in other important oncogenic pathways.

### Methods of Screening and Treatment

Accordingly, the invention provides methods and compositions for treating a subject having cancer. The invention also provides methods for determining a response to cancer therapy in a subject to be treated or being treated with a miR-34 therapeutic, including evaluating or predicting a therapeutic response.

The invention also provides for use of p21^{CIP1/WAF1}, PUMA, BAX, NOXA, PHLDA3, MDM2, or HDAC1, alone or in combination, as a predictive biomarker for miR-34 activity. The invention also provides for the use of p21^{CIP1/WAF1}, PUMA, BAX, NOXA, PHLDA3, MDM2, or HDAC1, alone or in combination, as a biomarker for determining a response to cancer therapy comprising administering a miR-34 therapeutic. In use, the miR-34 can be miR-34a, b, or c, or miR-449a, b, or c.

In some embodiments, the methods of treating a subject having cancer comprise screening the subject for the presence or absence of at least one biomarker prerequisite for miR-34 activity. In some embodiments, the methods comprise:

(a) screening the subject for the presence or absence of at least one prerequisite biomarker for miR-34 activity,

(b) administering a miR-34 therapeutic to the subject if the prerequisite biomarker(s) for miR-34 activity is determined to be present, and

(c) administering an alternative therapy to the subject if the miR-34 prerequisite biomarker(s) is absent,

thereby treating the subject.

In some embodiments, the prerequisite biomarker(s) can be p21^{CIP1/WAF1}, PUMA, BAX, NOXA, PHLDA3, MDM2, or HDAC1. For example, the subject can be screened for the presence or absence of p21^{CIP1/WAF1}, and a miR-34 therapeutic can be administered to the subject if p21^{CIP1/WAF1} is determined to be present. In some embodiments, a level of cellular proliferation decreases after administration of the miR-34 therapeutic. In some embodiments, the subject has been determined to have p21-positive cancer cells. For example, the subject has been determined to have p21-positive and p53-deficient cancer cells. For example, the subject can be determined to have cancer cells having a p21^{CIP1/WAF1} wild type (+/+) or heterozygous (+/−) gene and a homozygously inactivated (−/−) p53 gene. In another example, the subject can be determined to have cancer cells having a p21^{CIP1/WAF1} wild type (+/+) or heterozygous (+/−) gene and lacking functional p53 protein. In another example, the subject can be determined to have functional p21^{CIP1/WAF1} protein and non-functional or missing p53 protein. Examples of cancers that could be p21-positive and p53-deficient include p21-positive/p53-negative cancers, and cancers of various types, including non-small cell lung cancer (NSCLC), prostate cancer, bladder cancer (Koga et al., Jpn J Cancer Res 91(4):416-23 (2000)), esophageal cancer (Nakamura et al., Dis Esophagus 17(4):315-21 (2004); breast cancer (Thor et al., Breast Cancer Res Treat 61(1):33-43 (2000)), and stomach cancer (Xiangming et al., 148(2):181-8 (2000).

In some embodiments, the method of determining a response to cancer therapy in a subject being treated with a miR-34 therapeutic comprises:

(a) measuring a first level of at least one biomarker of miR-34 activity in the subject,

(b) administering a miR-34 therapeutic to the subject,

(c) measuring a second level of the biomarker(s) in the subject,

(d) further administering the miR-34 therapeutic to the subject if the second level relative to the first level indicates efficacy of the miR-34 therapeutic, and

(e) administering an alternative therapy to the subject if the second level relative to the first level indicates insufficient efficacy of the miR-34 therapeutic,

thereby determining the response of the subject to the cancer therapy and treating the subject.

The miR-34 therapeutic administered in steps (b) and (d) can be the same or different. The alternative therapy can be (i) a non-miR-34 microRNA therapy, (ii) a non-microRNA therapy, or (iii) any combination of (i) and (ii). For example, the alternative therapy can include at least one of miR-215 or miR-192. In some embodiments, determining the response can be determining that the treatment will take a longer time or a shorter time as compared to another treatment.

miR-34 Therapeutics

MicroRNAs (miRNAs) are small non-coding, naturally occurring RNA molecules that post-transcriptionally modulate gene expression and determine cell fate by regulating multiple gene products and cellular pathways.

It was previously demonstrated that miR-34 is involved with the regulation of numerous cell activities that represent intervention points for cancer therapy and for therapy of other diseases and disorders (U.S. Pat. Nos. 7,888,010 and 8,173,611, which are hereby incorporated by reference). miR-34 also functions as a tumor suppressor through its ability to regulate the expression of a numbers of key oncogenes (US Patent App. Pub. No. 2009/0227533, which is hereby incorporated by reference). Among the cancer-related genes that are regulated directly or indirectly by miR-34 are angiogenin, aurora kinase B, BCL10, BRCA1, BRCA2, BUB1, cyclin A2, cyclin D1, cyclin D3, CDK-4, CDK inhibitor 2C, FAS, forkhead box Ml, HDAC-1, c-Jun, MCAM, Mcl-1, c-Met, Myb L2, NF1, NF2, PI 3-kinase, polo-like kinase 1, R-RAS, SMAD3, TGF beta receptor, TPD52 tumor protein D52, and Wnt-7b. Thus, miR-34 governs the activity of proteins that are critical regulators of cell proliferation and survival. These targets are frequently deregulated in human cancer.

MicroRNA-34 replacement therapy has emerged as a promising approach to treat cancer and is currently in Phase I clinical trials. miR-34 is transcriptionally induced by p53. miR-34 also activates p53 in a positive feedback loop which previous literature suggested might be required for the miR-34 phenotype. However, mutations in the TP53 gene are the most common genetic changes found in human cancer, occurring occur in approximately half of all cancers.

A miR-34 therapy is a therapy that includes a miR-34 therapeutic. miR-34 therapies can include combination therapies that comprise a miR-34 therapeutic. For example, a miR-34 therapy can comprise a miR-34 therapeutic and further comprise another microRNA therapeutic, such as miR-215 (SEQ ID NO:1) or miR-192 (SEQ ID NO:2). In another example, a miR-34 therapy can comprise a miR-34 therapeutic and further comprise a non-microRNA therapy, such as chemotherapy, radiotherapy, or surgery.

A miR-34 therapeutic is an agent that increases amounts of miR-34 in a subject. In some embodiments, miR-34 therapeutics comprise human miR-34 and analogs thereof. miR-34 can include, but is not limited to a miR-34a, a miR-34b, a miR-34c, a miR-449a, a miR-449b, a miR-449c, a modified miR-34 nucleic acid, and any combinations thereof. As oligonucleotides, miR-34 therapeutics can be double stranded or single stranded. In some embodiments, miR-34 comprises the seed sequence of miR-34a (SEQ ID NO:3), miR-34b (SEQ ID NO:4), or miR-34c (SEQ ID NO:5) (Table 1). In some embodiments, miR-34 comprises the seed sequence of miR-449a (SEQ ID NO:6), miR-449b (SEQ ID NO:7), or miR-449c (SEQ ID NO:8) (Table 1). These microRNAs are well known in the art, and one of skill in the art would understand that they include the conventionally naturally occurring sequences (provided herein) and any chemically modified versions and sequence homologs thereof. In general, the miRNAs used are 17-25 nucleotides long, double stranded RNA molecules, either having two separate strands or a hairpin structure. One of the two strands, which is referred to as the “guide strand,” contains a sequence with is identical or substantially complementary to the seed sequence of the corresponding given miRNA. “Substantially complementary,” as used herein, means that at most 1 or 2 mismatches and/or deletions are allowed. In some embodiments, the guide strand is comprises a sequence which is at least 60%, 65%, 70%, 75%, 80%, 85%, 90%, 95%, 99%, or 100% homologous to the seed sequence. In some embodiments, the other strand (the “passenger strand”) comprises a sequence which is at least 60%, 65%, 70%, 75%, 80%, 85%, 90%, 95%, or 99% identical to the complement of the respective full length sequence provided herein. Various miR-34 therapeutics can be used, such as, for example, those described in U.S. Pat. No. 8,173,611, US Patent App. Pub. Nos. 2009/0227533 and 2012/0288933, which are incorporated by reference herein.

TABLE 1
miR-34 Family Regions. The underlined portions
of the sequences represent the seed regions.

| SEQ ID Number | Designation | Sequence |
|---|---|---|
| SEQ ID NO: 3 | hsa-miR-34a | UGGCAGUGUCUUAGCUGGUUGUU |
| SEQ ID NO: 4 | hsa-miR-34b | UAGGCAGUGUCAUUAGCUGAUUG |
| SEQ ID NO: 5 | hsa-miR-34c | AGGCAGUGUAGUUAGCUGAUUGC |
| SEQ ID NO: 6 | hsa-miR- | UGGCAGUGUAUUGUUAGCUGGU |
| | 449a | |
| SEQ ID NO: 7 | hsa-miR- | AGGCAGUGUAUUGUUAGCUGGC |
| | 449b | |
| SEQ ID NO: 8 | hsa-miR- | UAGGCAGUGUAUUGCUAGCGGCU |
| | 449c | GU |
| SEQ ID NO: 23 | consensus | *GGCAGUGU*UUAGCUG*UUG* |

Typically, miR-34 is formulated in liposomes such as, for example, those described in U.S. Pat. Nos. 7,858,117 and 7,371,404; US Patent App. Pub. Nos. 2009/0306194 and 2011/0009641. Other delivery technologies are available, including expression vectors, lipid or various ligand conjugates, polymer-based nanoparticles, etc.

A miR-34 therapeutic can also be chemically modified; for example, modified miR-34 may have a 5′ cap on the passenger strand (e.g., NH₂—(CH₂)₆—O—) and/or mismatch at the first and second nucleotide of the same strand. Other possible chemical modifications can include backbone modifications (e.g., phosphorothioate, morpholinos), ribose modifications (e.g., 2′-OMe, 2′-Me, 2′-F, 2′-4′-locked/bridged sugars (e.g., LNA, ENA, UNA) as well as nucleobase modifications (see e.g., Peacock et al., 2011. J Am Chem Soc., 133(24):9200-9203). In certain embodiments, miR-34 has modifications as described in U.S. Pat. No. 7,960,359 and US Patent App. Pub. Nos. 2012/0276627 and 2012/0288933.

miR-34 therapeutics can be administered in various ways, for example topically, enterally or parenterally. Specifically, parenteral delivery can involve intravenous or subcutaneous administration. For example, miR-34 could be administered intravenously as a slow-bolus injection at doses ranging between about 0.001-10.0 mg/kg per dose, for example, 0.01-3.0, 0.025-1.0 or 0.25-0.5 mg/kg per dose, with one, two, three or more doses per week for 2, 4, 6, 8 weeks or longer as necessary.

### Subject/Cancers

In methods of the inventions, a subject can be an animal, for example, a mammal, such as a human. In some embodiments, the subject can be a human having cancer, suspected of having cancer, or susceptible to cancer. A subject susceptible to cancer may have either historical (e.g., prior cancer), environmental (cigarette smoking, excessive sunlight exposure, exposure to certain chemicals) or genetic (e.g., Lynch syndrome) indicators of susceptibility. Exemplary cancers include, without limitation, lung cancer (non-small cell lung cancer (NSCLC), e.g., adenocarcinoma, squamous cell carcinoma, and large cell carcinoma), pancreatic cancer, cancer in the liver, hepatocellular carcinoma, breast cancer, colorectal cancer, head and neck cancers, prostate cancer, brain cancer, stomach cancer, bladder cancer, esophageal cancer, or colon cancer. In some embodiments, the subject has been determined to have p21^{CIP1/WAF1}-positive cancer cells. In some embodiments, the subject can have p53-deficient cancer cells. In certain examples, the subject has been determined to have cancer cells that are both p21^{CIP1/WAF1}-positive and p53-deficient. For example, the subject can be determined to have cancer cells having a p21^{CIP1/WAF1} wild type (+/+) or heterozygous (+/−) gene and a homozygously inactivated (−/−) p53 gene. In another example, the subject can be determined to have cancer cells having a p21^{CIP1/WAF1} wild type (+/+) or heterozygous (+/−) gene and lacking functional p53 protein. In another example, the subject can be determined to have functional p21^{CIP1/WAF1} protein and non-functional or missing p53 protein. In some embodiments, the subject may have failed a prior first-line therapy, such as a non-miR-34 miRNA therapy or a non-miRNA therapy. For example, the subject may have experienced one or more significant adverse side effects to the first-line therapy or the first-line therapy may not have treated cancer cells in the subject. For example, administration of a first-line miRNA therapy, such as miR-192 or miR-215, does not stop cancer cell proliferation in the subject. In some embodiments, the subject can have primary or metastatic cancer, or cancer of stage I, II, III, or IV.

### Biomarkers

A biomarker of miR-34 activity is a proxy for miR-34 activity, e.g., a molecule whose expression or activity indicates that miR-34 is functionally active. For example, a biomarker of miR-34 activity can be a direct target of miR-34, such as HDAC1, a molecule that interacts with miR-34, or a molecule that is induced by miR-34. FIG. 1 shows miR-34 targets, some of which are in common with p53, and some of which are separate, that are biomarkers of miR-34 activity. Expression levels or activity of these biomarkers can be measured using assays, as discussed further below. In some aspects, a biomarker of miR-34 activity can be used as a predictive marker of miR-34 activity, i.e., to predict how a subject will respond to a miR-34 therapeutic. In some aspects, changes in expression levels or activity levels of biomarker of miR-34 activity can be used to determine how well a subject is responding to a miR-34 therapy. Biomarkers of miR-34 activity include, without limitation, p21^{CIP1/WAF1}(CDKN1A) (OMIM#116899) (SEQ ID NO:9), p53 (OMIM#191170) (SEQ ID NO:10), PUMA (OMIM#605854) (SEQ ID NO:11), BAX (OMIM#600040) (SEQ ID NO:12), NOXA (OMIM#604959) (SEQ ID NO:13), PHLDA3 (OMIM#607054) (SEQ ID NO:14), MDM2 (OMIM#164785) (SEQ ID NO:15), and HDAC1 (OMIM#601241) (SEQ ID NO:16) described in detail below. Examples of these biomarkers and their sequence listings are discussed in US Patent App. Pub. Nos. 2008/0274956, 2010/0292085, and 2009/0298054 and WO 2000/075184, all of which are incorporated herein by reference.

A prerequisite biomarker of miR-34 activity is a biomarker that, if absent, renders the subject not responsive to the miR-34 therapy, i.e., a biomarker of response to miR-34. Some biomarkers of miR-34 activity, for example p21^{CIP1/WAF1}(CDKN1A), are prerequisite biomarkers of miR-34 activity, while other biomarkers of miR-34 activity, for example p53, are not necessary for miR-34 activity. For example, a biomarker of miR-34 activity can show that miR-34 is active in a subject, but nonetheless there may not be a therapeutic benefit in the subject if a prerequisite biomarker of miR-34 activity is not present. Prerequisite biomarkers of miR-34 activity can include, without limitation, p21^{CIP1/WAF1}(CDKN1A), PUMA, BAX, NOXA, PHLDA3, MDM2, and HDAC1. If the expression level or activity level is equal to or above a reference level, the biomarker is determined to be present, otherwise the biomarker is determined to be absent. The reference level can vary depending on the type of cell or the subject. For example, the reference level can be a minimum level detectable by a particular assay. For example, the reference level can be equal to or within a normal range of expression or activity for the biomarker in a subject. For example, a biomarker can be determined to be present if the expression level or activity level is within 1%, 2%, 3%, 4%, 5%, 10%, 15%, 20%, 25%, 30%, 33%, 40%, 50%, 60%, 70%, 75%, 80%, or 90% of the reference level.

A biomarker can indicate efficacy of a miR-34 therapeutic if there is a relative change between a first level of the biomarker expression or activity and a second level of the biomarker expression or activity. A relative change between the first level and the second level of at least 3%, 4%, 5%, 10%, 20%, 25%, 30%, 40%, 50%, 60%, 70%, 75%, 80%, 90%, 95%, 99%, or 100% can indicate efficacy of a miR-34 therapeutic. Depending on the biomarker, the second level can be higher or lower than the first level to indicate efficacy. For example, the second level can be at least 50% greater than the first level for biomarkers of miR-34 activity that are expected to go up in response to a miR-34 therapeutic, such as p21. In other examples, the second level can be at least 50% lower than the first level for biomarkers of miR-34 activity that are expected to go down in response to a miR-34 therapeutic, such as HDAC1.

p21^{CIP1/WAF1}

Human p21^{CIP1/WAF1} is localized to the chromosome 6p21.2 on a gene CDKN1A that encodes a cyclin dependent kinase (CDK) inhibitor. The protein p21^{CIP1/WAF1} was first described in 1992 (Xiong et al., Cell 71:505-514(1992)). p21^{CIP1/WAF1} is a potent tumor suppressor otherwise known to be transcriptionally regulated by p53 and necessary for the p53 response. The primary functions of p21^{CIP1/WAF1} involve cell cycle arrest by inhibiting cyclin-dependent kinases (CDKs) and blockage of DNA synthesis by binding to proliferating cell nuclear antigen (PCNA) (Abbas et al., Nat Rev Cancer 9(6):400-14 (2009)). However, p21^{CIP1/WAF1} can also inhibit other oncogenic pathways, including those regulated by WNT4, STAT3, MYC and TERT (Abbas et al., Nat Rev Cancer 9(6):400-14 (2009)).

p21^{CIP1/WAF1} belongs to the Cip/Kip family of CKIs (p21^{CIP1/WAF1}, p27^{KIP1} and p57^{KIP2}) which are involved in the regulation of the activity of the cyclin/CDK complex and have been shown to negatively regulate the process of cyclin-mediated cell cycle progression through inhibition of the CDKs (US Patent App. Pub. No. 2005/043262). Alterations in p21^{CIP1/WAF1} may adversely affect the regulation of cellular proliferation and increase the susceptibility to cancer. As such p21^{CIP1/WAF1} polymorphisms have been observed in various human cancers.

A p21^{CIP1/WAF1}-positive cancer cell is a cancer cell having a detectable expression level or activity level of functional p21^{CIP1/WAF1}. For example, a p21^{CIP1/WAF1}-positive cancer cell can be a cancer cell that has wild-type p21^{CIP1/WAF1} activity or expression levels. For example, p21^{CIP1/WAF1} activity levels can be detected by measuring cell proliferation or by using an siRNA directed against p21^{CIP1/WAF1}.

p53

As discussed above, the tumor suppressor TP53 (p53) transcriptionally induces the expression of all three miR-34 family, but has a high mutation rate in cancer. A p53-normal cancer cell has p53 wild type expression and activity. A p53-deficient cancer cell has a lower expression level and/or activity level than p53-normal cells. Cancer cells that have heterozygous or homozygous TP53 mutations are p53-deficient cancer cells. For example, cells that have dominant negative mutations are p53-deficient cancer cells. In some embodiments, p53-deficient cancer cells do not have any endogenous p53. In some embodiments, p53-deficient cancer cells do not have functional p53. Lung cancer (non-small cell lung cancer (NSCLC), e.g., adenocarcinoma, squamous cell carcinoma, and large cell carcinoma), pancreatic cancer, cancer in the liver, hepatocellular carcinoma, breast cancer, colorectal cancer, head and neck cancers, prostate, brain, stomach, bladder, esophageal, or colon cancer cells can be p53-deficient cancer cells. The level of expression or activity of p53 protein or mRNA can be measured by an assay. For example, the expression level can be the level of functional p53 protein. For example, genomic tests can measure dominant negative mutations.

### Other Biomarkers

PUMA, BAX, NOXA, PHLDA3, MDM2 and HDAC1 are other exemplary biomarkers of miR-34 activity. PUMA, BAX, NOXA, PHLDA3, and MDM2 are upregulated by miR-34. HDAC1, a well-known drug target, is downregulated in cells transfected with miR-34a and has been implicated in transcriptional regulation. The HDAC1 transcript has a miR-34 binding site in its 3′-untranslated region (UTR) that is directly targeted by miR-34, which then represses HDAC1.

### Assays

Various assays can be used to measure parameters in performing the methods of the present invention. In some embodiments, assays can directly or indirectly measure expression or activity of genes, mRNA, and proteins. In some embodiments, activity assays can be used to indirectly measure expression levels of genes, mRNA, or proteins. Commons assays include, without limitation, proliferation assays, quantitative reverse-transcriptase PCR (qRT-PCR), luciferase reporter assays, ELISA, and Western analysis. In some embodiments, assays can be performed using biological samples, such as blood or tissue samples from the subject, such as tumor biopsy samples, for example. In some embodiments, other parameters can be measured as indicators of response to treatment. For example, tumor size, rate of apoptosis, cellular proliferation, hair loss, etc. can be measured.

### Alternative Cancer Therapies

The methods of the present invention can use cancer therapies other than miR-34 therapeutics, such as non-miR-34 microRNA therapies or non-microRNA therapies. These therapies can be used in conjunction with miR-34 therapeutics as miR-34 therapies or they can be used as alternative therapies. Cancer therapies that exclude miR-34 therapeutics will be referred to herein as alternative therapies. For example, an alternative therapy can be used as a “first-line” therapy, i.e., prior to administering a miR-34 therapeutic. In some embodiments, the alternative therapies are microRNA therapies. For example, the alternative therapy can be a microRNA, such as a miR-215 therapeutic, a miR-192 therapeutic, or a combination microRNA therapeutic that excludes miR-34. In some embodiments, the alternative therapies are non-microRNA therapies. Non-microRNA therapies include, without limitation, discontinued therapy, chemotherapy, radiation, surgery, palliative therapy, targeted therapies (e.g., an EGFR-TKI (for example, erlotinib, geftinib, etc.), bevacizumab, crizotinib), etc.

### General

In the methods of the invention, “administering” is not limited to any particular delivery system, and may include, without limitation, parenteral (including subcutaneous, intravenous, intramedullary, intraarticular, intramuscular, or intraperitoneal), rectal, topical, transdermal, or oral (for example, in capsules, suspensions, or tablets). Administration to an individual may occur in a single dose or in repeat administrations, and in any of a variety of physiologically acceptable salt forms, and/or with an acceptable pharmaceutical carrier and/or additive as part of a pharmaceutical composition. Physiologically acceptable salt forms and standard pharmaceutical formulation techniques, dosages, and excipients are well known to persons skilled in the art (see, e.g., Physicians' Desk Reference (PDR®) 2005, 59^{th} ed., Medical Economics Company, 2004; and Remington: The Science and Practice of Pharmacy, eds. Gennado et al., 21^{st} ed., Lippincott, Williams & Wilkins, 2005).

Additionally, effective dosages achieved in one animal may be extrapolated for use in another animal, including humans, using conversion factors known in the art (see, e.g., Freireich et al., Cancer Chemother Reports 50(4):219-244 (1966); Table 2 for equivalent surface area dosage factors).

TABLE 2
Equivalent Surface Area Dosage Factors.

From:

| | Mouse | Rat | Monkey | Dog | Human |
|---|---|---|---|---|---|
| To: | (20 g) | (150 g) | (3.5 kg) | (8 kg) | (60 kg) |
| Mouse | 1 | 0.5 | 0.25 | 0.17 | 0.08 |
| Rat | 2 | 1 | 0.5 | 0.25 | 0.14 |
| Monkey | 4 | 2 | 1 | 0.6 | 0.33 |
| Dog | 6 | 4 | 1.7 | 1 | 0.5 |
| Human | 12 | 7 | 3 | 2 | 1 |

The following examples provide illustrative embodiments of the invention. One of ordinary skill in the art would recognize the numerous modifications and variations that may be performed without altering the spirit or scope of the present invention. Such modifications and variations are encompassed within the scope of the invention. The Examples do not in any way limit the invention.

### EXAMPLES

### Example 1

### Materials and Methods

Cell Culture, Oligos and Proliferation Assays.

Isogenic cancer cells derived from the MCF10A breast cancer and the SW48, HCT116, DLD-1 and RKO colorectal cancer cell lines were obtained from Horizon Discovery Ltd (Cambridge, UK). See Table 3. Synthetic miRNA mimics and siRNAs were purchased from Life Technologies (Ambion, Austin, Tex.). For stimulation of the TP53 pathway, cells were pre-treated with 10 μM etoposide for 28 hours, and RNA was harvested for qRT-PCR analysis. Optimal transfection conditions using Lipofectamine® 2000 (Invitrogen) or RNAiMAX™ (Invitrogen) were determined for each cell line using an siRNA against EG5, a spindle protein required for proliferation. Reverse transfections were done in duplicates or triplicates. Briefly, 5 μl of oligo solution in RNAse-free water was added to 20 μl of Opti-MEM® per well containing Lipofectamine® 2000 (SW48, MCF10A, DLD-1, HCT116) or RNAiMAX (RKO). The mixture was incubated for 20 min at room temperature to form lipid-RNA complexes. Then, 75 μl of cells suspended in medium were added to reach a final concentration of 6,000-10,000 cells per well, depending on the growth rate of each cell line. After approximately 18 hours, the supernatant was removed and replaced with fresh media. Cellular proliferation was determined using AlamarBlue® (Invitrogen, Carlsbad, Calif.) 3-4 days post transfection. The AlamarBlue® substrate is metabolically converted into a fluorescent product in viable cells that is proportional to the number of living cells. Non-linear regression and EC50 values were calculated using the Graphpad (Prism) software. All EC50 values were within the 95% confidence interval (P<0.05) of the regression trendline. EC50 values used here were defined as the half-maximal miRNA activity.

TABLE 3
TP53 genotypes of isogenic cancer cell lines

| Cell Line | TP53 Genotype | Allele 1 | Allele 2 | Other Mutations* |
|---|---|---|---|---|
| SW48 +/+ | +/+ | WT WT | WT | CTNNB1, EGFR, FBXW7 |
| SW48 +/− | +/− | Inactivated | Inactivated | |
| SW48 −/− | −/− | | Inactivated | |
| HCT116 +/+ | +/+ | WT WT | WT | CTNNB1, CDKN2A, KRAS, |
| HCT116 +/− | +/− | Inactivated | Inactivated | MLH1, PIK3CA |
| HCT116 −/− | −/− | | Inactivated | |
| RKO +/+ | +/+ | WT WT | WT | BRAF, NF1, PIK3CA |
| RKO +/− | +/− | Inactivated | Inactivated | |
| RKO −/− | −/− | | Inactivated | |
| DLD-1 par | S241F/SIL | S241F WT | Silent | not documented |
| DLD-1 +/− | +/SIL | Inactivated | Silent | |
| DLD-1 −/− | −/SIL | | Silent | |
| MCF10A +/+ | +/+ | WT | WT | not documented |
| MCF10A −/− | −/− | Inactivated | Inactivated | |

*as reported by the Catalogue of Somatic Mutations in Cancer (COSMIC, www.sanger.ac.uk) database

Quantitative Reverse-Transcriptase PCR.

Total RNA from cultured isogenic cancer cell lines was isolated using the mirVANA™ PARIS™ RNA isolation kit (Ambion) following the manufacturer's instructions. For qRT-PCR detection of miRNAs, 10 ng of total RNA and miRNA-specific RT-primers for each of hsa-miR-34a, hsa-miR-215, hsa-miR-192, hsa-miR-194, hsa-miR-34b, and hsa-miR-34c (Assay IDs 000426, 000518, 000491, 000492, 002102, 000428; TaqMan® miRNA Assay, Applied Biosystems) were heat-denatured at 70° C. for 2 min and reverse-transcribed using MMLV reverse transcriptase (cat. no. 28025-021, Invitrogen). miRNA expression levels were determined by PCR using Platinum Taq Polymerase reagents (Invitrogen) and the ABI Prism 7900 SDS instrument (Applied Biosystems). PCR reactions were performed by heating samples to 95° C. for 1 min, followed by incubating the samples at 95° C. for 5 sec, and 60° C. for 30 sec during multiple cycles. The house-keeping miRNAs miR-191 and miR-103 (Assay IDs 002299 and 000439) were amplified as internal references to adjust for well-to-well RNA input variances. Raw Ct values were normalized to the geometric mean of house-keeping miRNAs CTs and expressed as fold-differences relative to those in untreated, miR-NC or mock-transfected cells.

For detection of human mRNAs, cDNA was generated using 10 ng total RNA with random decamers (AM5722G, Ambion). Gene-specific amplification was carried out using multiple Taqman Gene Expression Assays (Invitrogen). mRNA levels of house-keeping GAPDH and cyclophilin A (Taqman®; Invitrogen) were used as loading controls. Raw Cts were normalized to those of house-keeping mRNAs and analyzed as described above.

Site-Directed Mutagenesis.

Human HDAC1 3′-UTR Lenti-reporter-Luciferase vector (pLenti-UTR-Luc HDAC1, HDAC1 wt) encoding the luciferase reporter fused to the entire 3′-UTR of human HDAC1 was purchased from Applied Biological Materials, Inc. Two rounds of mutagenesis were performed to introduce 6 point mutations in the miR-34 binding site of HDAC1 3′-UTR (HDAC1 mut). For site-directed mutagenesis, the QuikChange XL Site-Directed Mutagenesis Kit (Agilent Technologies) was used following the manufacturer's instructions. In the first round, the following primers were used: SEQ ID NO:17 (CCTCAAGTGA GCCAAGAAAC AATAACTGCC CTCTGTCTGTC) and SEQ ID NO:18 (GACAGACAGA GGGCAGTTAT TGTTTCTTGG CTCACTTGAGG). A positive clone was verified by sequencing (UT Austin) and used as a template for the second round of mutagenesis using the following primers: SEQ ID NO:19 (GGCCTCAAG TGAGCCAAAA ATAAATAACT GCCCTCTGTC TGTC) and SEQ ID NO:20 (GACAGACAGA GGGCAGTTAT TTATTTTTGG CTCACTTGAG GCC). All vectors used in transfections were verified by sequencing.

Luciferase Reporter Assays.

SW48^{-/-} and H1299 cells were reverse transfected with 1 nM or 10 nM miR-34a, respectively, in 96-well plate using lipofectamine2000 (Life Technology). As controls, cells were transfected with miR-NC at the same concentrations. The next day, cells were forward transfected with each 100 ng of HDAC1 wt or HDAC1 mut luciferase plasmids. After 48 hours, cell lysates were prepared and quantified using the BCA system from Pierce (Thermo Scientific). Luminescence was determined using the POLARStar OPTIMA plate reader (BMG Labtech) and the Luciferase Assay System (Promega). Luminescence was normalized to total protein input.

Western Analysis.

200,000 SW48 and RKO cells were seeded in 6-well plates and reverse-transfected with miRNA mimics and siRNAs in 6-well plate using 2.5 μl Lipofectamine2000 or RNAiMax. After 3 days, cell lysate were collected in RIPA buffer (Cell Signaling), and protein concentrations were measured using the BCA assay kit from Thermo Scientific. Each 2.5 μg of total cell lysate was loaded on 12% SDS-PAGE, and then transferred to a PVDF membrane. The membrane was blotted with primary antibody specific for p21, HDAC1, c-MET and actin (Cell Signaling) overnight at 4° C. The membrane was washed in 1× phosphate-buffered saline (PBS) containing 0.2% Tween™-20 and incubated with a horseradish peroxidase-conjugated secondary antibody at room temperature for 1 hour. After washing with 1×PBS containing 0.2% Tween™-20, the membrane was incubated with ECL detection reagent (Thermo Scientific) and protein bands were visualized using the AFP X-ray film developer (AFP Image Corp.)

Human Tissue Samples.

NSCLC tumor samples and the corresponding normal adjacent tissues (NATs) were purchased from ProteoGenex and the National Disease Research Interchange. HDAC1 mRNA and miR-34a levels were determined by qRT-PCR and expressed as relative expression between each tumor and NAT pair. Linear regression was calculated using GraphPad.

Statistical Analysis.

Statistical analyses were done using the Excel and Graphpad (Prism) software. Averages and standard deviations were calculated from duplicate or triplicate experiments. P values were generated by 2-tailed Student's t-test or F test as indicated in the figure legends.

### Example 2

### Inhibition of Cancer Cell Proliferation by miR-34 is Independent of TP53

Isogenic cells used in this study were derived from the MCF10A breast cancer and the colorectal carcinoma cell lines SW48, HCT116, RKO and DLD-1 (Table 3). In these cells, TP53 is either wild-type (+/+), heterozygous (+/−) or homozygously inactivated (−/−) (Sur et al., Proc Natl Acad Sci USA 106(10):3964-9 (2009)). Parental DLD-1 cells (DLD-1^{par}) do not express a functional TP53 protein due to the S241F/SIL TP53 genotype in which one allele is mutated and the other is epitopically silenced. Therefore, DLD-1^{+/SIL} cells (+/−), in which the point mutation has been corrected by site-directed mutagenesis, serves as the DLD-1 reference line with intact TP53 (Sur et al., Proc Natl Acad Sci USA 106(10):3964-9 (2009)). Each non-isogenic cell line displays mutations in other tumor suppressor genes and oncogenes which may influence the inhibitory effects of miRNAs (Table 3).

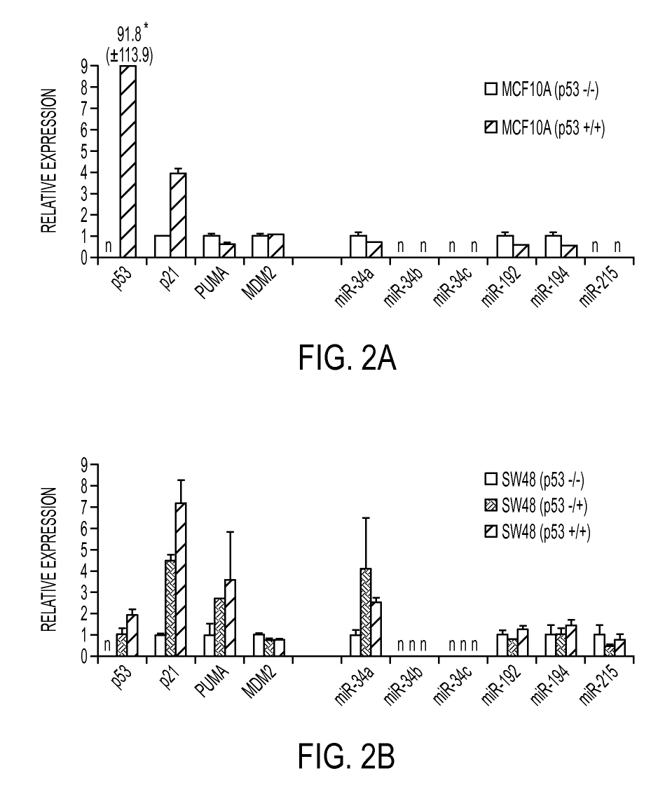

To confirm the serial inactivation of TP53 in isogenic cell lines, TP53 response was induced by exposing the cells to the DNA-damaging agent etoposide for 28 hours and collected total RNA. A quantitative reverse-transcriptase PCR (qRT-PCR) analysis showed an allele-dependent increase in TP53 mRNA and TP53-regulated target genes according to their genotype (FIGS. 2A-2E). TP53 mRNA was not detectable in TP53^{-/-} cells. Increased mRNA levels of TP53-regulated genes are similar to published data (Brady et al., Cell 145(4):571-83 (2011)) and varied between cell lines, presumably due to cell-type specific regulation of these genes. Likewise, the induction of TP53-regulated miRNAs, miR-34a/b/c, miR-192, miR-194 and miR-215 was dependent on the cell line all cell lines but DLD-1^{+/-} lacked miR-34b/c expression, and miR-215 was solely detectable in SW48 and DLD-1 cells.

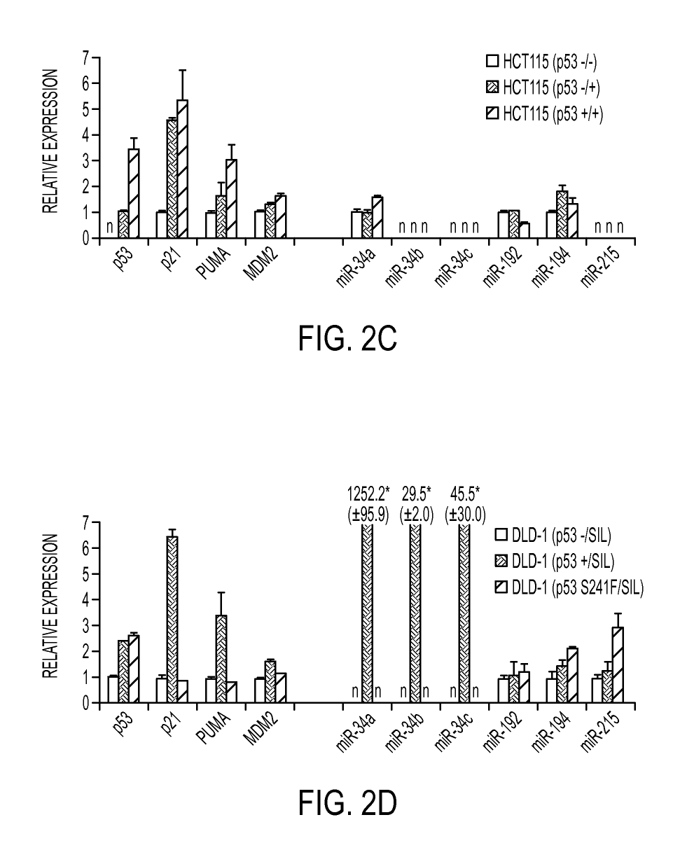

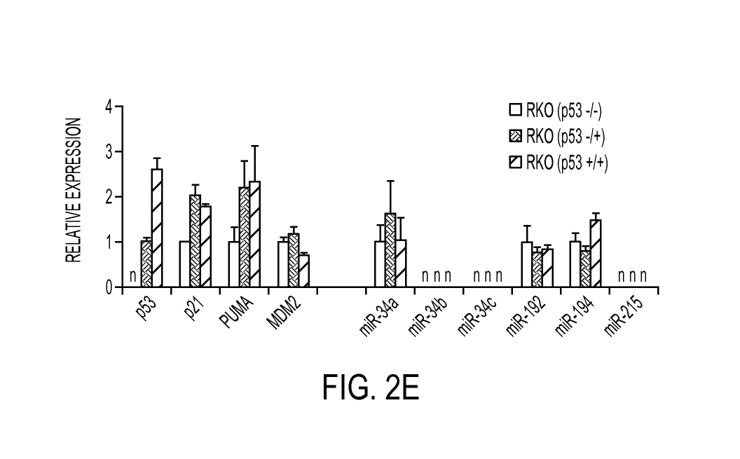

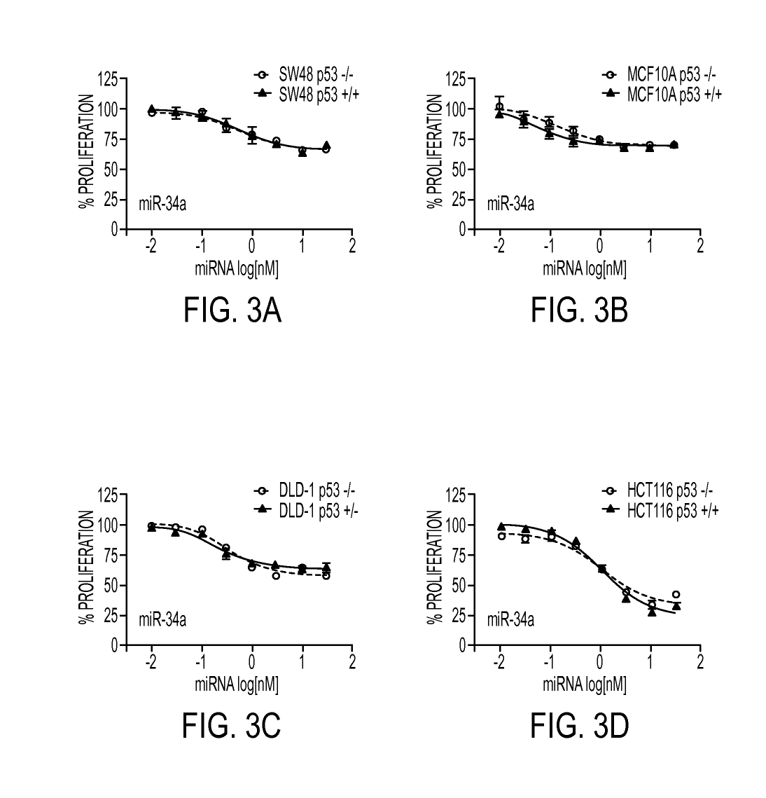

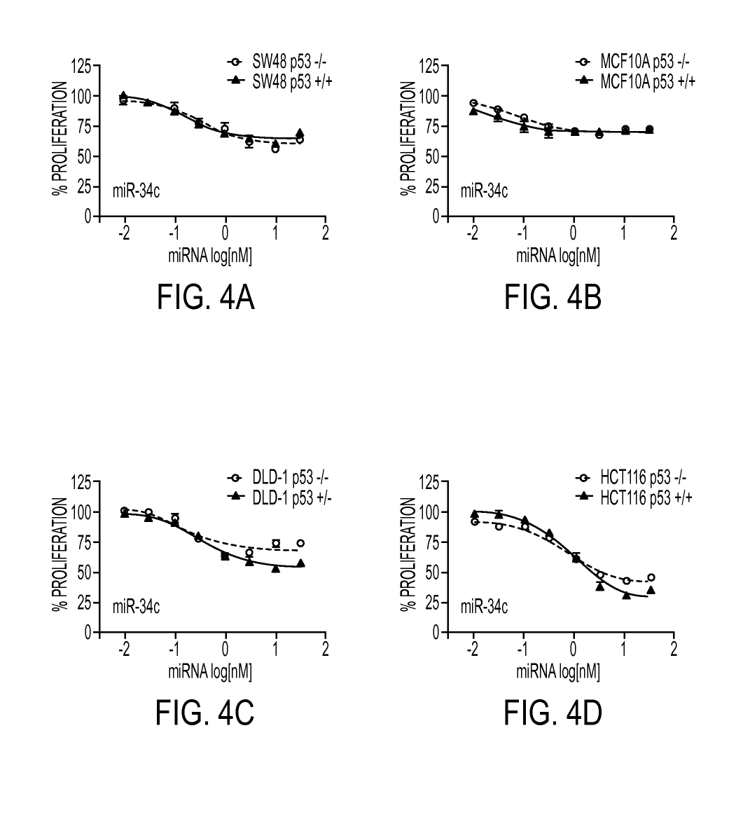

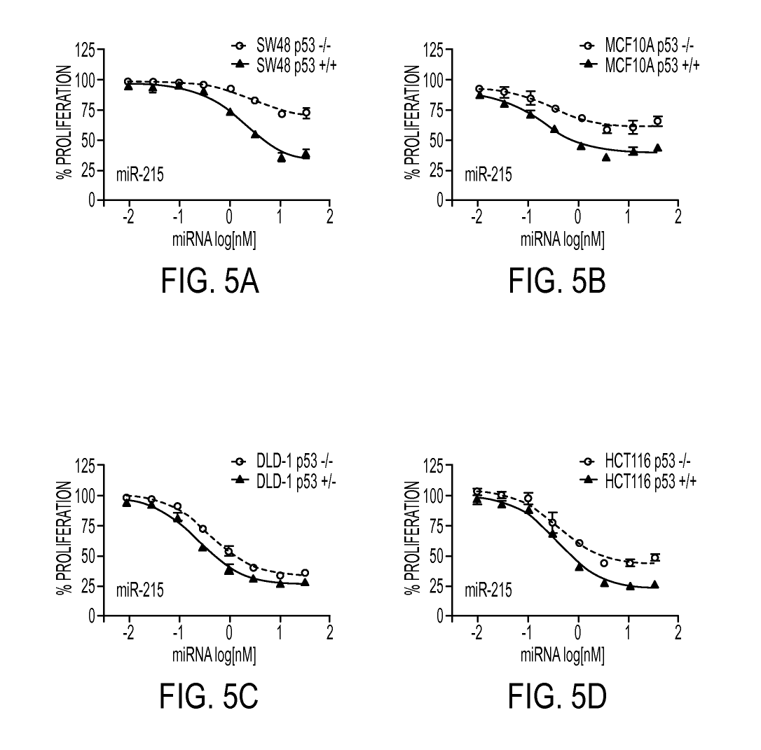

Isogenic cells were transfected with mimics of miR-34a, miR-34c, miR-192, miR-194 and miR-215. The miRNAs were used in a serial dilution to generate dose-response curves and to calculate EC50 values. As negative controls, mock-transfected cells and cells transfected with a miRNA carrying a scrambled sequence were used (miR-NC). After 3-4 days of incubation, cellular proliferation was assessed using AlamarBlue®. As shown in FIG. 3, miRNAs mimics inhibited cellular proliferation by ˜40-80% compared to controls. TP53 enhanced the ability of miR-215 and miR-192 to inhibit cancer cells and was greatest in MCF10A and SW48 cells with EC50 values ˜28-35-fold lower compared to TP53^{+/+} cells (Table 4). In contrast, the inhibitory activity of miR-34a and miR-34c was the same in TP53-positive and TP53-negative cells (FIGS. 3A-6D, Table 4). RKO cells showed greater inhibition in the absence of TP53, further demonstrating that TP53 is not a prerequisite for the miR-34-induced phenotype (FIGS. 7A-7B). Interestingly, in the presence of intact TP53, the maximal inhibitory activity of miR-215/192 was greater than the maximal activity of miR-34ac, suggesting that miR-215/192 function in the TP53 positive feedback loop and take advantage of ancillary pathways exclusively regulated by TP53.

TABLE 4
EC50* values of miRNAs in isogenic cancer cells.

| | SW48 | SW48 | |
|---|---|---|---|
| miRNA | p53 −/− | p53 +/+ | EC50^{TP53 wt}:EC50^{TP53-/- ‡} |
| miR-34a | 0.59 | 0.62 | 1.0 |
| miR-34c | 0.38 | 0.40 | 0.9 |
| miR-215 | 40.21 | 1.50 | 26.9 |
| miR-192 | 21.56 | 0.76 | 28.3 |
| | MCF10A | MCF10A | |
| | p53 −/− | p53 +/+ | EC50^{TP53 wt}:EC50^{TP53-/- ‡} |
| miR-34a | 0.11 | 0.04 | 2.7 |
| miR-34c | 0.10 | 0.04 | 2.9 |
| miR-215 | 5.42 | 0.17 | 31.8 |
| miR-192 | 6.79 | 0.19 | 35.3 |
| | DLD-1 | DLD-1 | |
| | p53 −/− | p53 +/− | EC50^{TP53 wt}:EC50^{TP53-/- ‡} |
| miR-34a | 0.39 | 0.39 | 1.0 |
| miR-34c | 1.34 | 0.38 | 3.6 |
| miR-215 | 0.69 | 0.26 | 2.7 |
| miR-192 | 0.63 | 0.22 | 2.9 |
| | HCT116 | HCT116 | |
| | p53 −/− | p53 +/+ | EC50^{TP53 wt}:EC50^{TP53-/- ‡} |
| miR-34a | 1.03 | 1.02 | 1.0 |
| miR-34c | 0.86 | 0.83 | 1.0 |
| miR-215 | 1.07 | 0.31 | 3.5 |
| miR-192 | 1.10 | 0.50 | 2.2 |

*EC50 values were generated with the Prism software and were within the 95% confidence interval of the trendline (P < 0.05). Values are expressed in nM.
^{‡} Ratios indicate fold-differences of EC50 values in TP53-positive and TP53-negative cells.

### Example 3

### miR-34a, but not miR-215, Induces TP53-Regulated Genes in the Absence of TP53

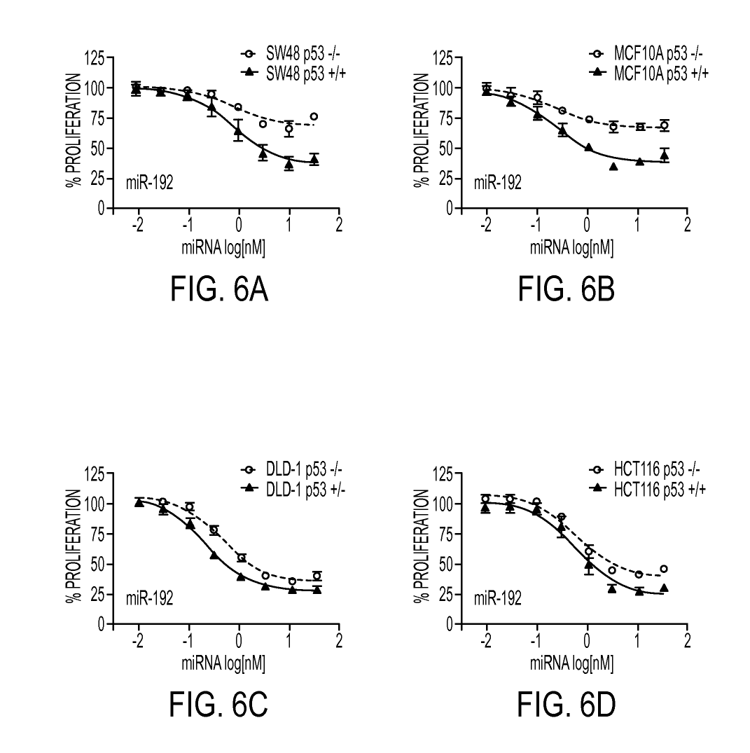

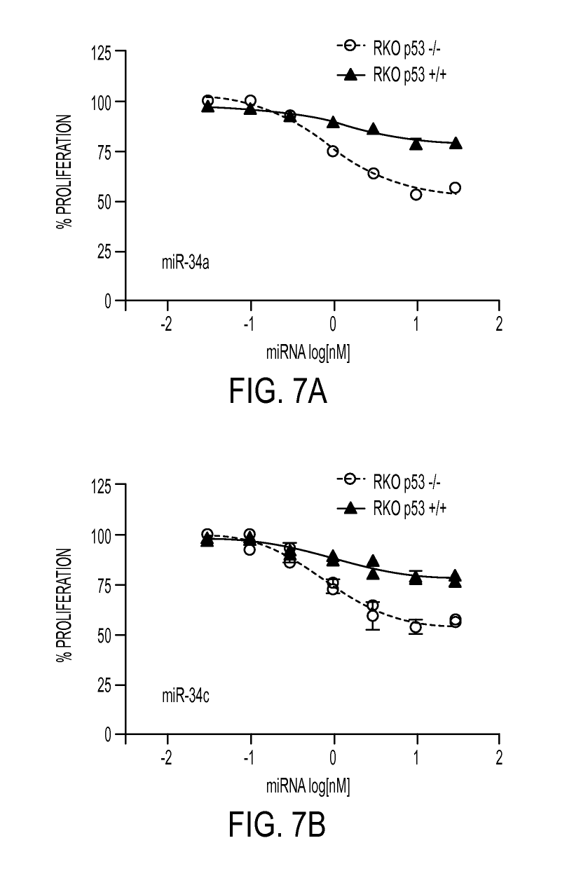

To understand the miR-34-induced phenotype in TP53-positive and TP53-deficient cells, expression levels of genes involved in the TP53/miR-34 axis were determined. One possible explanation for the TP53-independent effects is that these cells do not express endogenous SIRT1 or MDM4. However, as confirmed by qRT-PCR, both TP53^{+/+} and TP53^{-/-} cells carry detectable SIRT1 and MDM4 mRNA levels, suggesting that the TP53-independent phenotype is not due to an absence of these gene products (FIG. 8). Rather, both mRNAs were reduced in cells transfected with miR-34, in accordance with experimental data showing that SIRT1 and MDM4 are directly targeted by this miRNA (Yamakuchi et al., Proc Natl Acad Sci USA 105(36):13421-6 (2008); Mandke et al., PLoS One 7(8):e42034 (2012)). Similarly, MET, a miR-34a target, and BCL2, a miR-215 target, were specifically downregulated in cells transfected by the respective miRNAs (FIG. 8). TP53 mRNA levels were not detectable in SW48^{-/-} cells in accord with its defined genotype. In SW48^{+/+} cells, TP53 mRNA levels were constant and is in agreement with the hypothesis that the positive feedback loop to TP53 by these miRNAs does not require TP53 de novo synthesis but occurs post-transcriptionally. This is further corroborated by the observation showing that miR-215 induces the expression of p21^{CIP1/WAF1} (p21, CDKN1A) in TP53-positive cells, but fails to do so in TP53-deficient cells. Unexpectedly, miR-34a was able to induce p21, PUMA and MDM2 not only in TP53⁺⁴ cells, but also in TP53-deficient cells (FIG. 8). To ensure that this phenomenon is not specific to SW48, we tested all other isogenic cancer cells transfected with miR-34a and extended the analysis to other genes transcriptionally regulated by TP53. These include the pro-apoptotic proteins BAX and NOXA, as well as the tumor suppressor PHLDA3, a PH domain-only protein that functions as a negative regulator of AKT/PKB (Kawase et al., Cell 136:535-50 (2009)). In agreement with the data from SW48 cells, miR-34a induced an accumulation of the six transcripts not only in the presence, but also in the absence of functional TP53 (FIGS. 9A and 9B).

### Example 4

### HDAC1 is a Direct Target of miR-34a

A plausible explanation for the TP53-independent up-regulation of p21^{CIP1/WAF1} is a potential involvement of other TP53 family members, TP63 and TP73. Both proteins play roles distinct from TP53; however, they also control a set of genes that overlaps with that of TP53. Endogenous mRNA levels of TP63 and TP73 were measured in TP53-wild-type and TP53-deficient cells that had been transfected with miR-34a. However, none of the cells showed detectable levels TP63 or TP73 suggesting that an involvement of these gene products is unlikely (data not shown).

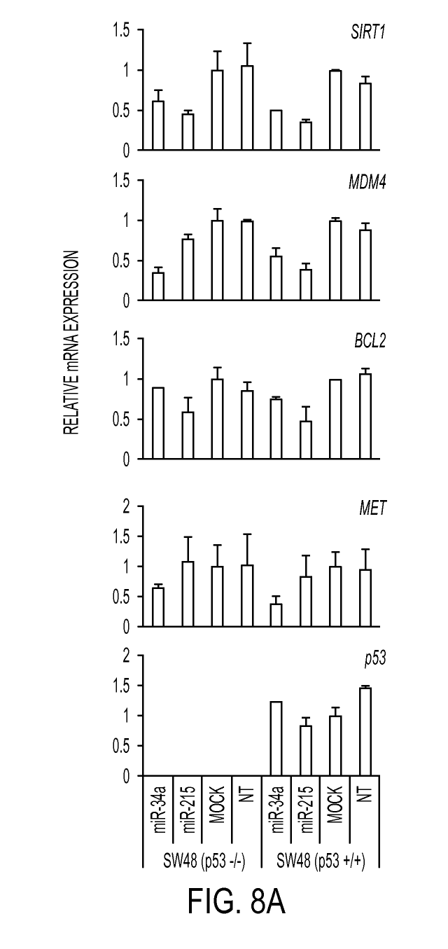

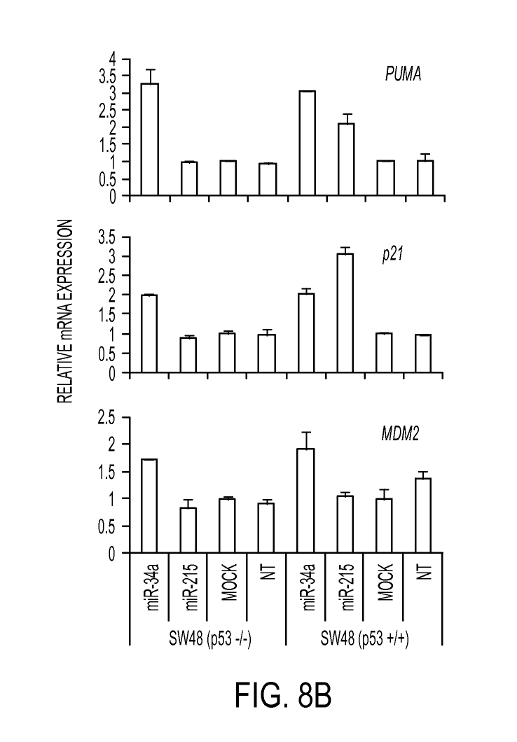

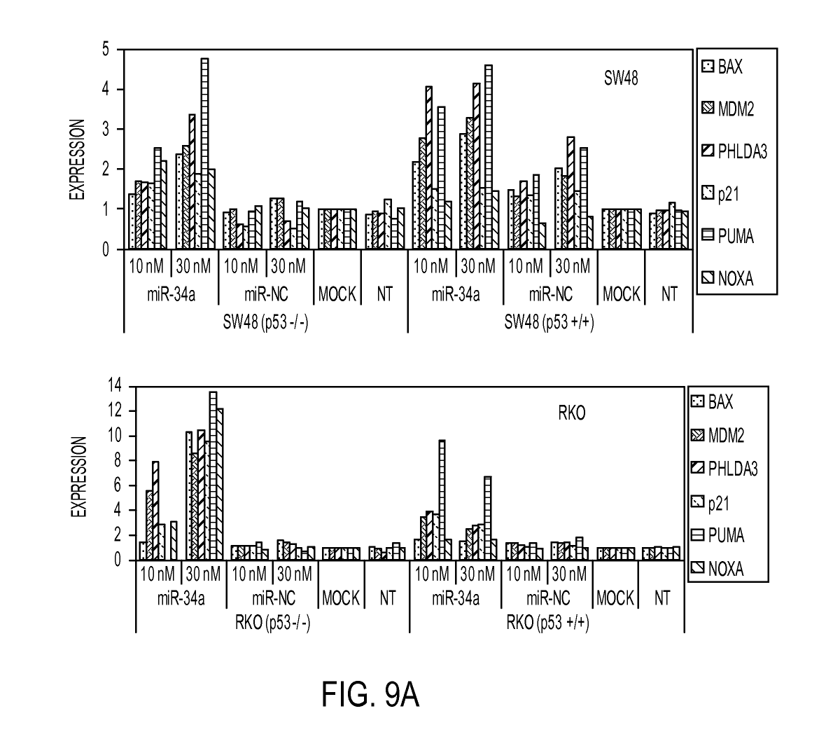

Next, regulatory mechanisms that are independent of TP53 and nuclear regulators that can control p21^{CIP1/WAF1} expression were analyzed. One candidate of interest was HDAC1 because it has a putative miR-34a binding site in its 3′UTR (FIG. 10), is downregulated in cells transfected with miR-34a (FIG. 11), and has been implicated in the transcriptional regulation of p21^{CIP1/WAF1} in the absence of TP53 (Lagger et al., Mol Cell Biol 23(8):2669-79 (2003)). To establish whether HDAC1 is directly repressed by miR-34a, miR-34a was examined to determine whether it can repress a luciferase reporter that is fused to the entire HDAC1 3′UTR (SEQ ID NO:21). This reporter was transiently expressed in two cell lines that lack endogenous miR-34a. Then, cells were transfected with miR-34a (SEQ ID NO:3) or miR-215 (SEQ ID NO:1), the latter of which is not predicted to repress HDAC1 and was used as a negative control. As shown in FIG. 12A, transfection of miR-34a diminished luminescence by ˜50% in both cell lines relative to controls. This repression was completely abolished upon mutation of the miR-34a binding site (SEQ ID NO:22) (FIG. 12A), suggesting that the HDAC1 3′UTR is directly targeted by miR-34a at this site. To further evaluate if the miR-34a-dependent repression of HDAC1 is reflected in human tumor specimens, we examined a cohort of 14 non-small cell lung cancer samples previously used to document reduced miR-34a expression levels (Wiggins et al., Cancer Res 70(14):5923-30 (2010)). Tumor HDAC1 mRNA and miR-34a levels were determined by qRT-PCR and normalized to the levels in their respective normal adjacent tissues. An analysis by the Pearson's method showed a statistically significant inverse correlation between HDAC1 mRNA and miR-34a levels (FIG. 12B), supporting a role for miR-34a in the regulation of HDAC1 in human tumors.

### Example 5

### Inhibition of HDAC1 Mimics the miR-34a Phenotype

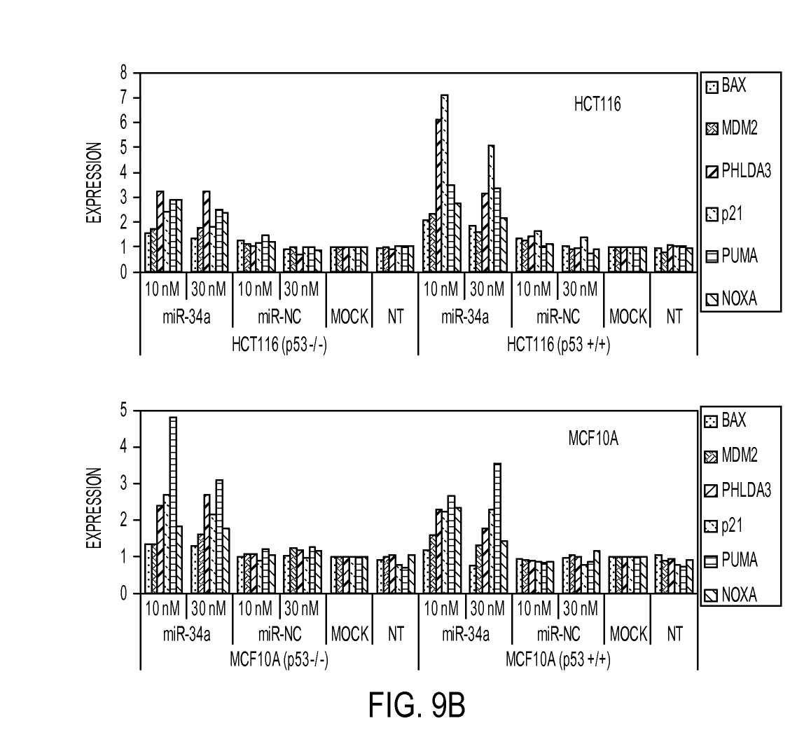

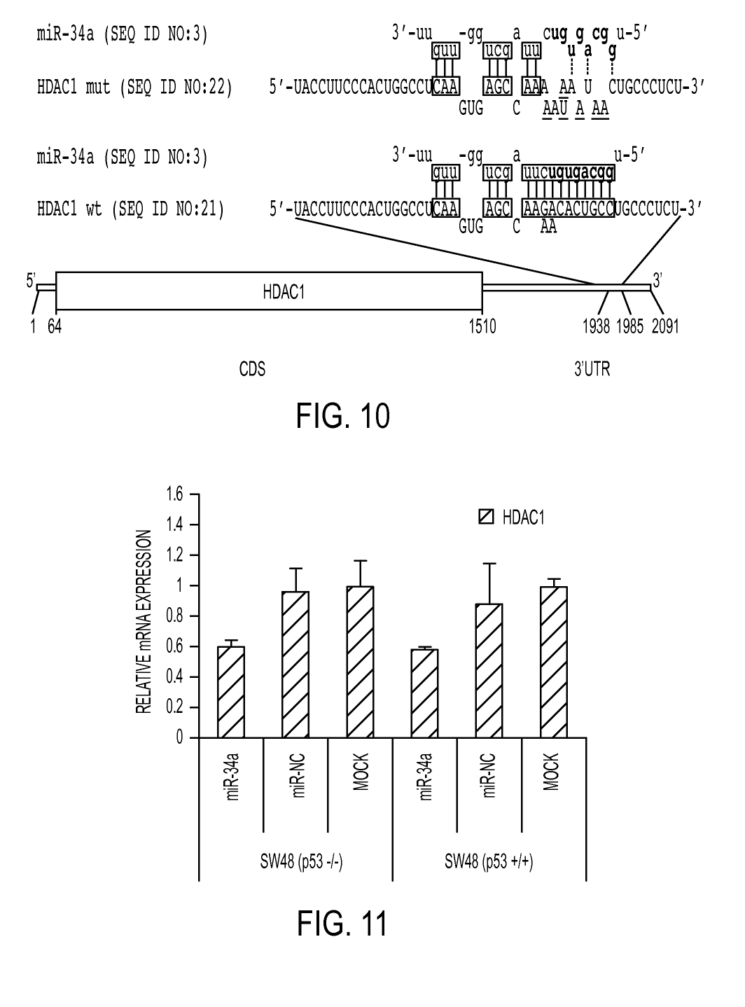

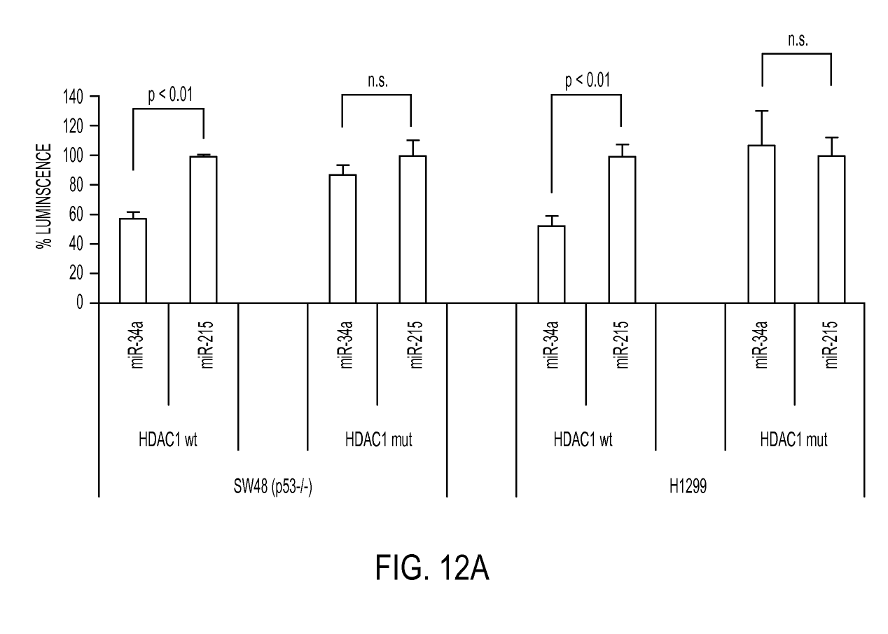

Previous results implicated HDAC1 in the regulation of the p21^{CIP1/WAF1} gene. For instance, HDAC1-deficient embryonic stem cells show elevated levels of p21^{CIP1/WAF1}, and inhibition of HDAC1 using the HDAC inhibitor trichostatin A (TSA) can induce p21^{CIP1/WAF1} expression in the absence of TP53 (Sowa et al., 1997; Lagger et al., 2002). To confirm the TP53-independent induction of p21^{CIP1/WAF1} upon depletion of HDAC1, we transfected TP53-negative cells with an siRNA directed against HDAC1 and evaluated cell lysates by Western blotting. The results were compared to cells transfected with miR-34a or miR-215. Two cell lines were tested and included mock- and miR-NC treated cells as negative controls. As expected, Met was solely downregulated in cells transfected with miR-34a, and HDAC1 protein was reduced by both miR-34a and the HDAC1 siRNA (FIGS. 13-14). Of note, both oligonucleotides induced a marked increase of p21^{CIP1/WAF1} protein expression in these cells. This observation was in stark contrast to miR-215 that failed to induce p21^{CIP1/WAF1} in TP53-deficient cells. However, transfection of miR-215 into TP53-positive cells led to an increase of p21^{CIP1/WAF1} protein in TP53-positive cells (FIG. 15) in accord with the hypothesis that the miR-215-dependent induction of p21^{CIP1/WAF1} is mediated by TP53 as a result of the positive feedback loop from miR-215 to TP53 (Picchiori et al., Cancer Cell 18(4):367-81 (2011)).

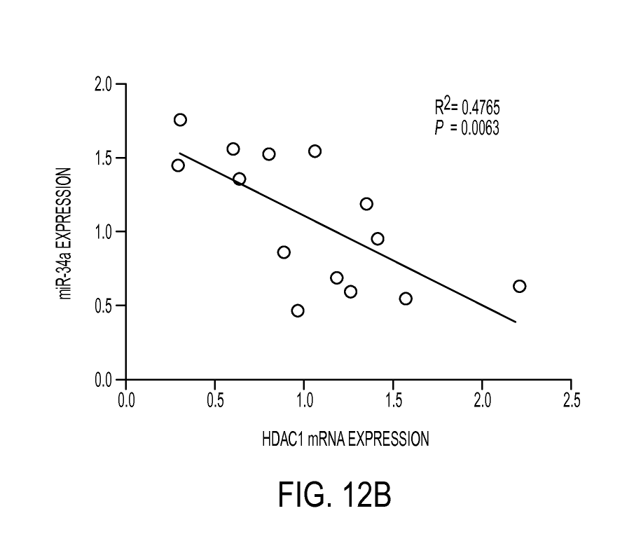

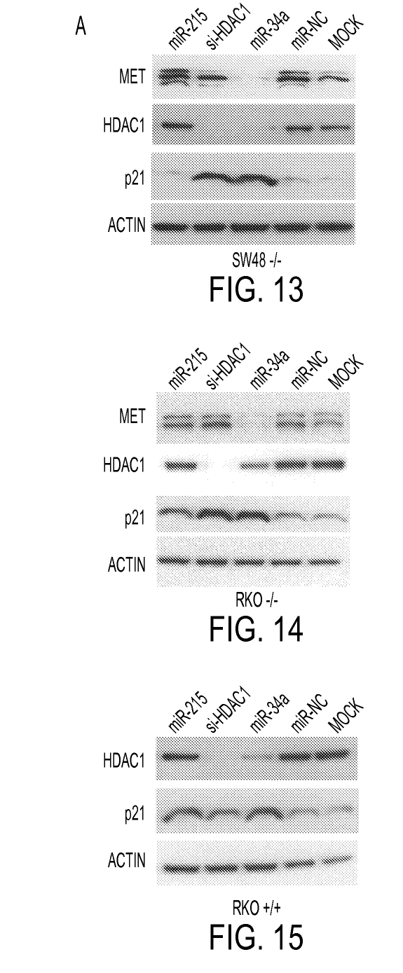

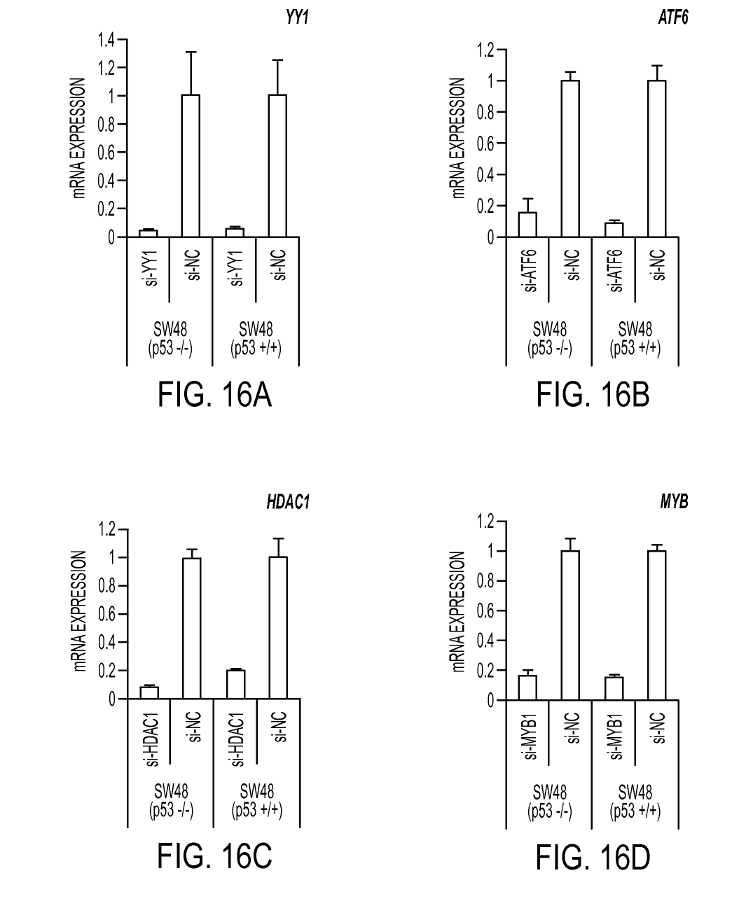

To explore whether inhibition of HDAC1 can mimic the miR-34a phenotype, we measured the proliferation effects of an siRNA against HDAC1 in both TP53-positive and TP53-deficient SW48 cells. Cells were also transfected with a series of other siRNAs directed against gene products that can antagonize TP53 function. These genes include YY1, MDM4 and SIRT1, as well as a few others that are either validated or predicted miR-34a targets and were repressed in miR-34a-transfected cells (data not shown). Transient transfection of siRNAs led to >80% knock-down of target mRNAs as confirmed by qRT-PCR (FIGS. 16A-16G). As controls, cells were also transfected with miR-34a and miR-215. We sought to identify siRNAs that yield a level of cancer cell inhibition that is similar in both cell lines. As expected, miR-34a equally inhibited SW48^{+/+} and SW48^{-/-} cells, and the activity of miR-215 was dependent on TP53 (FIG. 17). Most siRNAs failed to reduce cellular proliferation in either cell type, including the siRNA against SIRT1 and YY1. Knock-down of MDM4 was able to inhibit proliferation of SW48^{+/+} cells but had no effect in SW48^{-/-} cells. This is reminiscent of the miR-215 phenotype and confirms the role of MDM4 in modulating TP53 transactivation rather than DNA regulation (Toledo et al., 2006). In contrast, knock-down of HDAC1 inhibited cancer cell growth that—similarly to miR-34a—was the same in both isogenic cell lines. Similar results were obtained from cells treated with trichostatin A (FIGS. 18A-18B) further corroborating a role for HDAC1 in mediating a miR-34a response through p21^{CIP1/WAF1}.

### Example 6

### Depletion of p21 Interferes with miR-34a-Induced Inhibition of Cancer Cell Proliferation

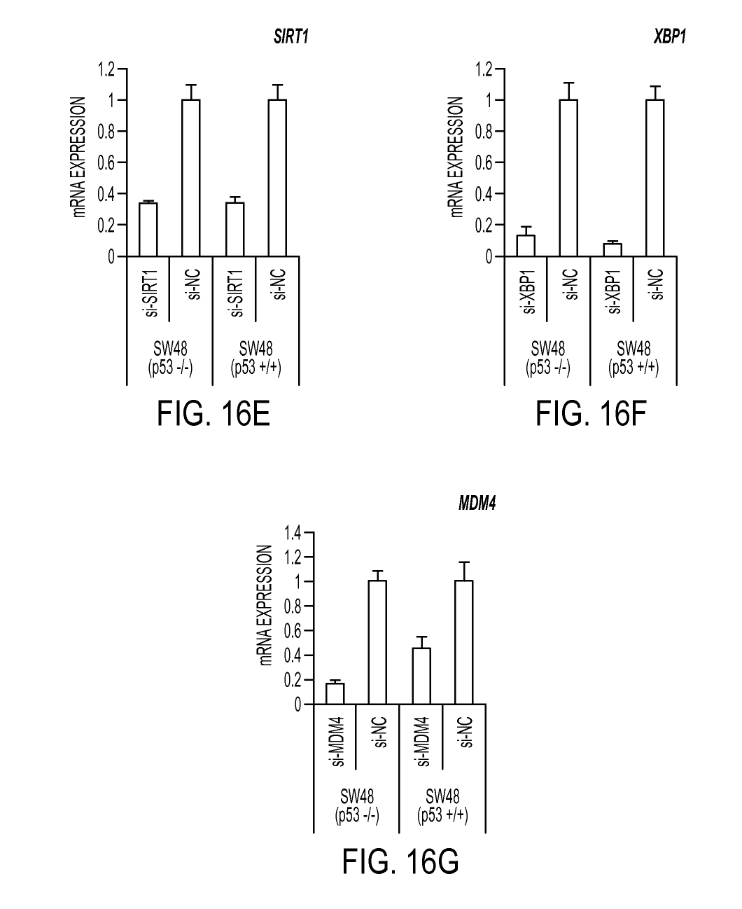

The dose-response data generated in various cell lines suggest that p21^{CIP1/WAF1} expression is a key event during miR-34a-induced inhibition of cancer cell proliferation. Expression levels of p21^{CIP1/WAF1} markedly correlated with the ability of miR-34a to inhibit TP53-positive and TP53-negative cells. For instance, the inhibitory activity of miR-34a was the same in MCF10A and SW48 cells and correlated with similar p21^{CIP1/WAF1} expression levels in both TP53^{-/-} and TP53^{+/+} cells (FIG. 3). HCT116 cells displayed greater p21^{CIP1/WAF1} mRNA levels in TP53^{+/+} compared to TP53^{-/-} cells, in accord with the slightly increased inhibitory activity of miR-34a in TP53⁺⁴ cells at higher miR-34a concentrations (30 nM, FIG. 3). In RKO cells, p21^{CIP1/WAF1} levels were higher in TP53^{-/-} cells and mirrored the greater inhibition of proliferation in RKO^{-/-} versus RKO cells (FIG. 7A-7B). The induction of p21^{CIP1/WAF1} was also evident at low miR-34a concentrations and inversely correlated with inhibition of cell proliferation (FIG. 19).

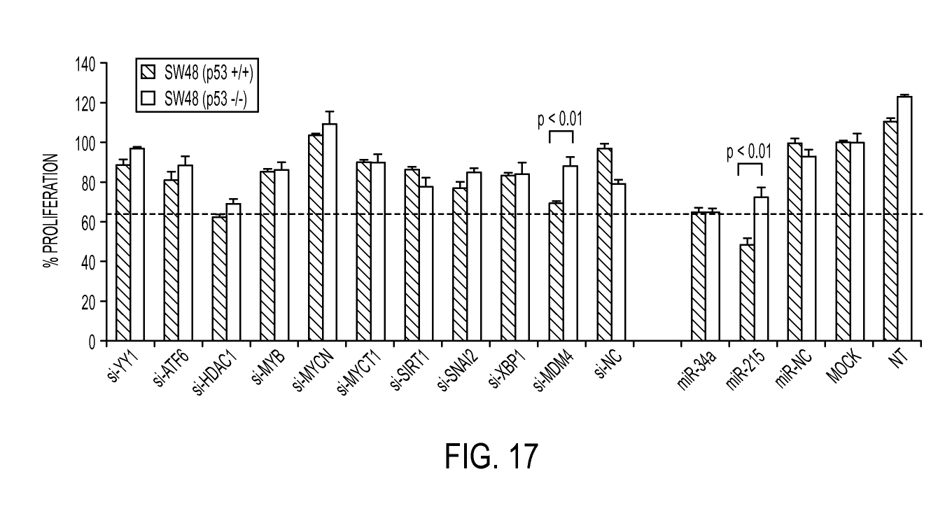

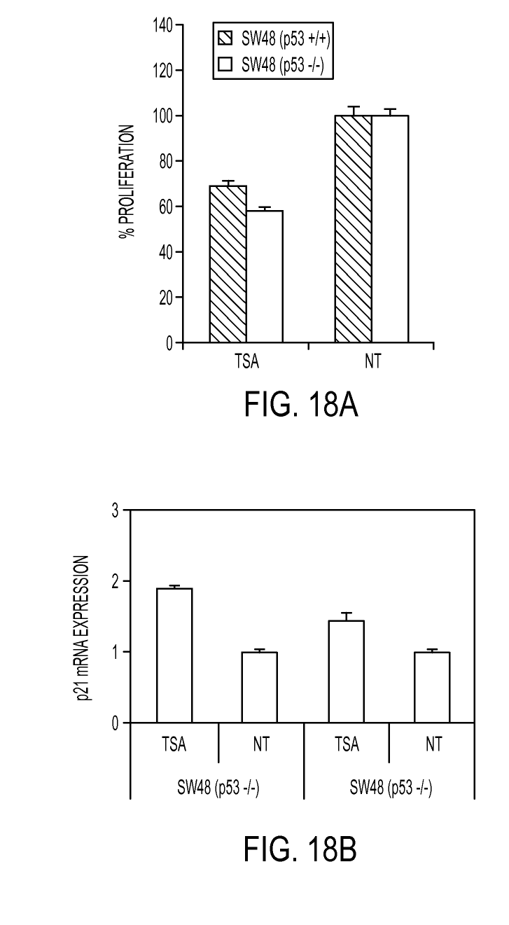

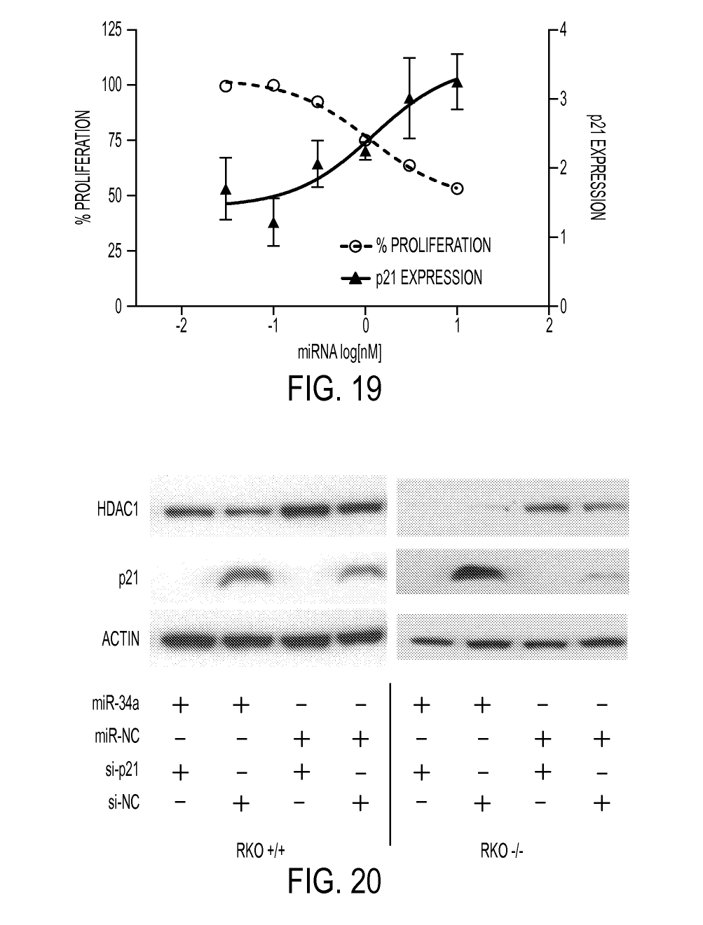

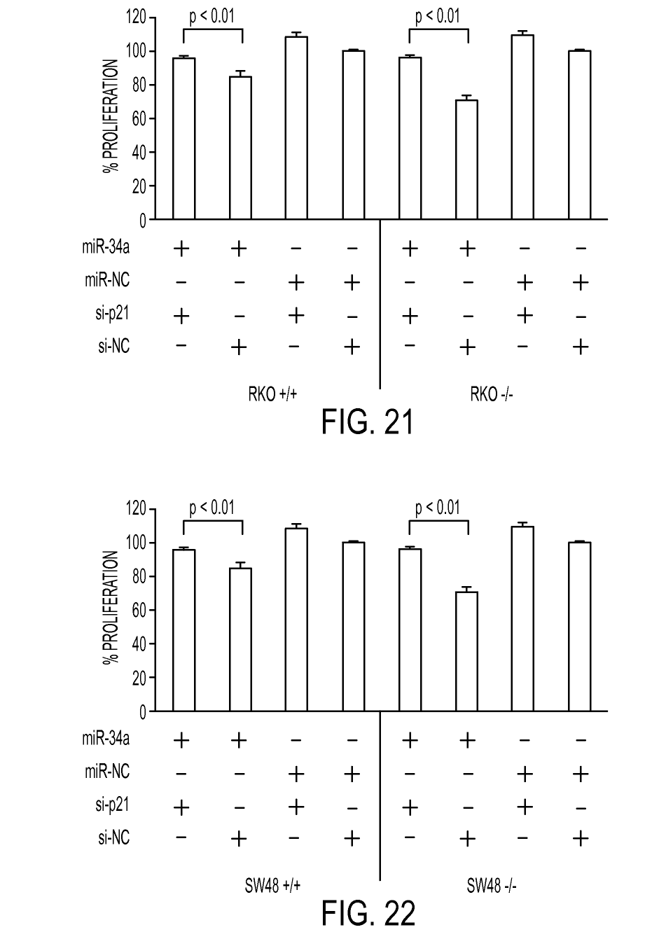

To address whether p21^{CIP1/WAF1} expression is required for the miR-34a-induced phenotype, we performed interference assays by co-transfecting cells with miR-34a and an siRNA directed against p21^{CIP1/WAF1}. As controls, cells were transfected with either miR-34a, miR-215 or miR-NC. Each miRNA was supplemented with negative control oligo such that the total amount of transfected RNA equals the one of the miR-34a/si-p21 combination. The downregulation of targeted genes was verified by Western analysis (FIG. 20). miR-34a alone reduced proliferation of RKO cells by ˜20-30% (FIG. 21). In contrast, the miR-34a/si-p21 combination had no effect on cancer cell proliferation and suggests that p21^{CIP1/WAF1} expression is indeed a necessary factor in mediating a miR-34 tumor suppressor response. The p21^{CIP1/WAF1}-dependent phenotype was reproducible in isogenic SW48 cancer cells (FIG. 22) and TP53-negative Hep3B hepatocarcinoma cells that lack p21^{CIP1/WAF1} (FIG. 23).

### Example 7

### Systemic Delivery of miR-34a Mimics In Vivo Induces the Expression of p21^{CIP1/WAF1} in Tumors

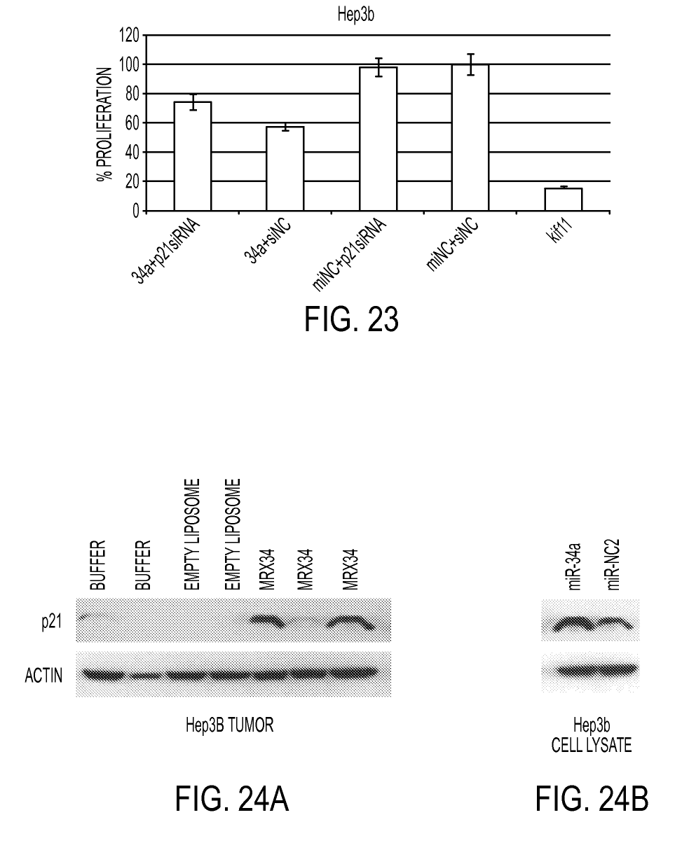

To address whether miR-34a can induce an upregulation of p21^{CIP1/WAF1} in tumor-bearing animals, Hep3B hepatocellular carcinoma cells were surgically implanted into the left lateral lobe of the liver in NODSCID mice. Hep3B cancer cells lack functional TP53. Approximately after 5 weeks when mice developed detectably large tumors, a single dose of a miR-34a mimic at a concentration of 1 mg per kg mouse body weight was administered by intravenous tail vein injection. The miR-34a mimic is a liposomal formulation containing a mimic of miR-34a. A dose of 1 mg/kg is equivalent to 20 μg miR-34a. After 24 hours, mice were sacrificed, and tumor tissues were collected used for protein lysate preparation. Lysates were probed by Western analysis to determine the expression of p21^{CIP1/WAF1} protein. As shown in FIG. 24A, two out of three MRX34-treated mice expressed p21^{CIP1/WAF1} at high levels. In contrast, none of the control tumors showed elevated expression of the p21^{CIP1/WAF1} protein. Similarly, Hep3B cells transiently transfected with miR-34a showed an upregulation of p21^{CIP1/WAF1}. FIG. 24B. The data suggest that a miR-34a-based therapy is able to induce p21^{CIP1/WAF1} in tumors that lack functional TP53.

It is claimed that:

1. A method of treating a subject having cancer, the method comprising:
(a) screening the subject for the presence or absence of at least one prerequisite biomarker for miR-34 activity;
(b) administering a miR-34 therapeutic to the subject if the prerequisite biomarker(s) for miR-34 activity is determined to be present; and
(c) administering an alternative therapy to the subject if the prerequisite biomarker(s) for miR-34 activity is determined to be absent,
thereby treating the subject.

2. The method of claim 1, wherein the subject has lung cancer, pancreatic cancer, cancer in the liver, hepatocellular carcinoma, breast cancer, colorectal cancer, head and neck cancer, prostate cancer, brain cancer, stomach cancer, bladder cancer, esophageal cancer, or colon cancer.

3. The method of claim 1, wherein the at least one prerequisite biomarker is selected from the group consisting of p21^{CIP1/WAF1}, PUMA, BAX, NOXA, PHLDA3, MDM2, and HDAC1.

4. The method of claim 1, wherein the at least one prerequisite biomarker comprises p21^{CIP1/WAF1}.

5. The method of claim 4, wherein the sole prerequisite biomarker for which the subject is screened is p21^{CIP1/WAF1}.

6. The method or use of claim 1, wherein miR-34 is miR-34a, miR-34b, miR-34-c, miR-449a, miR-449b, or miR-449c.

7. The method of claim 1, wherein the alternative therapy is discontinued therapy.

8. The method of claim 1, wherein the miR-34 therapeutic comprises miR-34 and further optionally comprises at least one of miR-215 and miR-192.

9. The method of claim 1, wherein the alternative therapy is a non-miR-34 microRNA therapy.

10. The method of claim 1, wherein the alternative therapy is selected from chemotherapy, radiotherapy and surgery, and further optionally comprises at least one of miR-215 and miR-192.

11. The method of claim 1, wherein the subject has been determined to have p21^{CIP1/WAF1}-positive cancer cells.

12. The method of claim 1, wherein the subject has been determined to have p21^{CIP1/WAF1}-positive cancer cells and p53-deficient cancer cells.

13. The method of claim 12, wherein the subject has been determined to have cancer cells lacking functional p53 protein.

14. The method of claim 13, wherein the subject has been determined to have cancer cells having a wild type or heterozygous p21^{CIP1/WAF1} gene.

15. The method of claim 1, wherein the subject has been determined to have p53 deficient cancer cells, the subject is screened for the presence or absence of p21^{CIP1/WAF1}, the miR-34 therapeutic is administered to the subject if p21^{CIP1/WAF1} is determined to be present, and the miR-34 therapeutic is discontinued if p21^{CIP1/WAF1} is determined to be absent.

16. A method of determining a response to cancer therapy in a subject being treated with a miR-34 therapeutic, the method comprising:
(a) measuring a first level of at least one biomarker of miR-34 activity in the subject;
(b) administering a miR-34 therapeutic to the subject;
(c) measuring a second level of the biomarker(s) in the subject;
(d) further administering a miR-34 therapeutic to the subject if the second level relative to the first level indicates efficacy of the miR-34 therapeutic; and
(e) administering an alternative therapy to the subject if the second level relative to the first level indicates insufficient efficacy of the miR-34 therapeutic,
thereby determining the response of the subject to cancer therapy.

17. The method of claim 16, wherein the subject has lung cancer, pancreatic cancer, cancer in the liver, hepatocellular carcinoma, breast cancer, colorectal cancer, head and neck cancer, prostate cancer, brain cancer, stomach cancer, bladder cancer, esophageal cancer, or colon cancer.

18. The method of claim 16, wherein a difference of 10% from the first level to the second level indicates efficacy of the miR-34 therapeutic.

19. The method of claim 16, wherein the miR-34 therapeutics in (b) and (d) are the same.

20. The method of claim 16, wherein the alternative therapy is a non-microRNA therapy.

21. The method of claim 16, wherein the non-microRNA therapy is discontinued therapy, chemotherapy, radiotherapy or surgery.

22. The method of claim 16, wherein the alternative therapy is a non-miR-34 microRNA therapy.

23. The method of claim 16, wherein the alternative therapy comprises at least one of miR-215 and miR-192.

24. The method of claim 16, wherein the at least one biomarker is a prerequisite biomarker of miR-34 activity.

25. The method of claim 24, wherein the at least one prerequisite biomarker is selected from the group consisting of p21^{CIP1/WAF1}, PUMA, BAX, NOXA, PHLDA3, and MDM2, and wherein efficacy is indicated if the second level is higher than the first level.

26. The method of claim 24, wherein the at least one prerequisite biomarker is HDAC1, and wherein efficacy is indicated if the second level is lower than the first level.

27. The method of claim 25, wherein the at least one prerequisite biomarker is p21^{CIP1/WAF1}, and wherein the alternative therapy is discontinued therapy.

28. A method of treating cancer cells in a subject, comprising:
selecting a subject that has p53-deficient and p21^{CIP1/WAF1}-positive cancer cells, and treating the subject with a miR-34 therapeutic.

29. The method of claim 28, wherein the subject has lung cancer, pancreatic cancer, cancer in the liver, hepatocellular carcinoma, breast cancer, colorectal cancer, head and neck cancer, prostate cancer, brain cancer, stomach cancer, bladder cancer, esophageal cancer, or colon cancer.

30. The method of claim 29, wherein the subject has bladder cancer, esophageal cancer, breast cancer, or stomach cancer.

31. The method of claim 1, wherein the subject has a leukemia, acute lymphoblastic leukemia (ALL), acute myelogenous leukemia (AML), acute lymphocytic leukemia, chronic lymphocytic leukemia (CLL), small lymphocytic lymphoma (SLL), chronic myelogenous leukemia (CML), acute monocytic leukemia (AMoL), a lymphoma, Hodgkin's lymphomas (all four subtypes), follicular lymphoma, B-cell lymphoma, non-Hodgkin's lymphomas (all subtypes), a myeloma, multiple myeloma, or a myelodystplastic syndrome.

32. The method of claim 1, wherein the prerequisite biomarker(s) is a DNA, mRNA, or protein.

33. The method of claim 32, wherein the DNA is a gene that is not silenced and that is free of any inactivating mutation(s).

34. The method of claim 1, wherein (i) the at least one prerequisite biomarker is selected from the group consisting of p21^{CIP1/WAF1} PUMA, BAX, NOXA, PHLDA3, and MDM2 and (ii) presence of the at least one prerequisite biomarker comprises an expression level or activity level that is equal to or above a reference level.

35. The method of claim 1, wherein (i) the at least one prerequisite biomarker is HDAC1;
and (ii) presence of the at least one prerequisite biomarker comprises an expression level or activity level that is equal to or below a reference level.


SEQUENCE LISTING (23 sequences)

SEQ ID NO: 1 - RNA, 21 nt, Homo sapiens
augaccuaugaauugacagac

SEQ ID NO: 2 - RNA, 21 nt, Homo sapiens
cugaccuaugaauugacagcc

SEQ ID NO: 3 - RNA, 23 nt, Homo sapiens
uggcagugucuuagcugguuguu

SEQ ID NO: 4 - RNA, 23 nt, Homo sapiens
uaggcagugucauuagcugauug

SEQ ID NO: 5 - RNA, 23 nt, Homo sapiens
aggcaguguaguuagcugauugc

SEQ ID NO: 6 - RNA, 22 nt, Homo sapiens
uggcaguguauuguuagcuggu

SEQ ID NO: 7 - RNA, 22 nt, Homo sapiens
aggcaguguauuguuagcuggc

SEQ ID NO: 8 - RNA, 25 nt, Homo sapiens
uaggcaguguauugcuagcggcugu

SEQ ID NO: 9 - DNA, 2175 bp, Homo sapiens
gttgtatatcagggccgcgctgagctgcgccagctgaggtgtgagcagctgccgaagtcagttccttgtggagccggagctgggcgcggattcgccgaggcaccgaggcactcagaggaggcgccatgtcagaaccggctggggatgtccgtcagaacccatgcggcagcaaggcctgccgccgcctcttcggcccagtggacagcgagcagctgagccgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaatccgcccacaggaagcctgcagtcctggaagcgcgagggcctcaaaggcccgctctacatcttctgccttagtctcagtttgtgtgtcttaattattatttgtgttttaatttaaacacctcctcatgtacataccctggccgccccctgccccccagcctctggcattagaattatttaaacaaaaactaggcggttgaatgagaggttcctaagagtgctgggcatttttattttatgaaatactatttaaagcctcctcatcccgtgttctccttttcctctctcccggaggttgggtgggccggcttcatgccagctacttcctcctccccacttgtccgctgggtggtaccctctggaggggtgtggctccttcccatcgctgtcacaggcggttatgaaattcaccccctttcctggacactcagacctgaattctttttcatttgagaagtaaacagatggcactttgaaggggcctcaccgagtgggggcatcatcaaaaactttggagtcccctcacctcctctaaggttgggcagggtgaccctgaagtgagcacagcctagggctgagctggggacctggtaccctcctggctcttgatacccccctctgtcttgtgaaggcagggggaaggtggggtcctggagcagaccaccccgcctgccctcatggcccctctgacctgcactggggagcccgtctcagtgttgagccttttccctctttggctcccctgtaccttttgaggagccccagctacccttcttctccagctgggctctgcaattcccctctgctgctgtccctcccccttgtcctttcccttcagtaccctctcagctccaggtggctctgaggtgcctgtcccacccccacccccagctcaatggactggaaggggaagggacacacaagaagaagggcaccctagttctacctcaggcagctcaagcagcgaccgccccctcctctagctgtgggggtgagggtcccatgtggtggcacaggcccccttgagtggggttatctctgtgttaggggtatatgatgggggagtagatctttctaggagggagacactggcccctcaaatcgtccagcgaccttcctcatccaccccatccctccccagttcattgcactttgattagcagcggaacaaggagtcagacattttaagatggtggcagtagaggctatggacagggcatgccacgtgggctcatatggggctgggagtagttgtctttcctggcactaacgttgagcccctggaggcactgaagtgcttagtgtacttggagtattggggtctgaccccaaacaccttccagctcctgtaacatactggcctggactgttttctctcggctccccatgtgtcctggttcccgtttctccacctagactgtaaacctctcgagggcagggaccacaccctgtactgttctgtgtctttcacagctcctcccacaatgctgaatatacagcaggtgctcaataaatgattcttagtgactttacttgtaaaaaaaaaaaaaaaaaa

SEQ ID NO: 10 - DNA, 2591 bp, Homo sapiens
gatgggattggggttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattggcagccagactgccttccgggtcactgccatggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccacctgtgtgtctgaggggtg

SEQ ID NO: 11 - DNA, 1839 bp, Homo sapiens
gaggcgattgcgattgggtgagacccagtaaggatggaaagtgtagaggagacaggaatccacggctttggaaaaaggaaggacaaaactcaccaaaccagagcagggcaggaagtaacaatgagaaactgaaaaagaaacggaatggaaagctatgagacaggatgaaatttggcatggggtctgcccaggcatgtccatgccaggtgcccagggctgcttccacgacgtgggtcccctgccagatttgtggccccagggagcgccatggcccgcgcacgccaggagggcagctccccggagcccgtagagggcctggcccgcgacggcccgcgccccttcccgctcggccgcctggtgccctcggcagtgtcctgcggcctctgcgagcccggcctggctgccgcccccgccgcccccaccctgctgcccgctgcctacctctgcgcccccaccgccccacccgccgtcaccgccgccctggggggttcccgctggcctgggggtccccgcagccggccccgaggcccgcgcccggacggtcctcagccctcgctctcgctggcggagcagcacctggagtcgcccgtgcccagcgccccgggggctctggcgggcggtcccacccaggcggccccgggagtccgcggggaggaggaacagtgggcccgggagatcggggcccagctgcggcggatggcggacgacctcaacgcacagtacgagcggcggagacaagaggagcagcagcggcaccgcccctcaccctggagggtcctgtacaatctcatcatgggactcctgcccttacccaggggccacagagcccccgagatggagcccaattaggtgcctgcacccgcccggtggacgtcagggactcggggggcaggcccctcccacctcctgacaccctggccagcgcgggggactttctctgcaccatgtagcatactggactcccagccctgcctgtcccgggggcgggccggggcagccactccagccccagcccagcctggggtgcactgacggagatgcggactcctgggtccctggccaagaagccaggagagggacggctgatggactcagcatcggaaggtggcggtgaccgagggggtggggactgagccgcccgcctctgccgcccaccaccatctcaggaaaggctgttgtgctggtgcccgttccagctgcaggggtgacactggggggggggggctctcctctcggtgctccttcactctgggcctggcctcaggcccctggtgcttccccccctcctcctgggagggggcccgtgaagagcaaatgagccaaacgtgaccactagcctcctggagccagagagtggggctcgtttgccggttgctccagcccggcgcccagccatcttccctgagccagccggcgggtggtgggcatgcctgcctcaccttcatcagggggtggccaggaggggcccagactgtgaatcctgtgctctgcccgtgaccgccccccgccccatcaatcccattgcataggtttagagagagcacgtgtgaccactggcattcatttggggggtgggagattttggctgaagccgccccagccttagtccccagggccaagcgctggggggaagacggggagtcagggagggggggaaatctcggaagagggaggagtctgggagtggggagggatggcccagcctgtaagatactgtatatgcgctgctgtagataccggaatgaattttctgtacatgtttggttaattttttttgtacatgatttttgtatgtttccttttcaataaaatcagattggaacagtggaaaaaaaaaaa

SEQ ID NO: 12 - DNA, 986 bp, Homo sapiens
tcacgtgacccgggcgcgctgcggccgcccgcgcggacccggcgagaggcggcggcgggagcggcggtgatggacgggtccggggagcagcccagaggcggggggcccaccagctctgagcagatcatgaagacaggggcccttttgcttcagggtttcatccaggatcgagcagggcgaatggggggggaggcacccgagctggccctggacccggtgcctcaggatgcgtccaccaagaagctgagcgagtgtctcaagcgcatcggggacgaactggacagtaacatggagctgcagaggatgattgccgccgtggacacagactccccccgagaggtctttttccgagtggcagctgacatgttttctgacggcaacttcaactggggccgggttgtcgcccttttctactttgccagcaaactggtgctcaaggctggcgtgaaatggcgtgatctgggctcactgcaacctctgcctcctgggttcaagcgattcacctgcctcagcatcccaaggagctgggattacaggccctgtgcaccaaggtgccggaactgatcagaaccatcatgggctggacattggacttcctccgggagcggctgttgggctggatccaagaccagggtggttgggacggcctcctctcctactttgggacgcccacgtggcagaccgtgaccatctttgtggcgggagtgctcaccgcctcactcaccatctggaagaagatgggctgaggcccccagctgccttggactgtgtttttcctccataaattatggcatttttctgggaggggtggggattgggggacgtgggcatttttcttacttttgtaattattggggggtgtggggaagagtggtcttgagggggtaataaacctccttcgggacacaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 13 - DNA, 1954 bp, Homo sapiens
actggacaaaagcgtggtctctggcgcggggatctcagagtttcccgggcactcaccgtgtgtagttggcatctccgcgcgtccggacacccgatcccagcatccctgcctgcaggactgttcgtgttcagctcgcgtcctgcagctgtccgaggtgctccagttggaggctgaggttcccgggctctgtagctgagtgggcggcggcaccggcggagatgcctgggaagaaggcgcgcaagaacgctcaaccgagccccgcgcgggctccagcagagctggaagtcgagtgtgctactcaactcaggagatttggagacaaactgaacttccggcagaaacttctgaatctgatatccaaactcttctgctcaggaacctgactgcatcaaaaacttgcatgaggggactccttcaaaagagttttctcaggaggtgcacgtttcatcaatttgaagaaagactgcattgtaattgagaggaatgtgaaggtgcattcatgggtgcccttggaaacggaagatggaatacatcaaagtgaatttctgttcaagttttcccagattatcattctttgggatgagagaacattataaaaccactttgtttattttaaagcaagaatggaagacccttgaaaataaagaagtaattattgacacatttcttttttacttagagaatcgttctagtgtttttgccgaagattaccgctggcctactgtgaagggagatgacctgtgattagactgggcggctggggagaaacagttcagtgcattgttgttgttgctgtttttggtgttttgcttttcagtgccaactcagcacattgtatatgattcggtttatacatattaccttgttataatgaaaaaactcattctgagaacactgaaatgttatactcagtgttgatttcttcggtcactacacaacgtaaaatcatttgtttcttttgactcaaattgtattgcttctgttcagatgatctttcattcaatgtgttcctgttgggcgttactagaaactatggaaaactggaaaataactttgaaaaaattggataaagtataggagggttacttggggccagtaaatcagtagactgaacattcaatataataaaagaacatggggattttgtataaccagggataataaaaagaaaaaagaagttaatttttaattgatgtttttgaaacttagtagaacaaatattcagaagtaacttgataagatatgaatgtttctaaagaagtttctaaaggttcggaaaatgctccttgtcacattagtgtgcatcctacaaaaagtgatctcttaatgtaaattaagaatattttcataattggaatatacttttcttaaaaaaaaggaacagttagttctcatctagaatgaaagttccatatatgcattggtgaatatatatgtatacacatacttacatacttatatgggtatctgtatagataatttgtattagagtattatatagcttcttagtagggtctcaagtaagtttcattttttttatctgggctatatacagtcctcaaataaataatgtcttgattttatttcagcaggaataattttatttattttgcctatttataattaaagtatttttctttagtttgaaaatgtgtattaaagttacatttttgagttacaagagtcttataactacttgaatttttagttaaaatgtcttaatgtaggttgtagtcactttagatggaaaattacctcacatctgttttcttcagtattacttaagattgtttatttagtggtagagagttttttttttcagcctagaggcagctattttaccatctggtatttatggtctaatttgtatttaaacatatgcacacatataaaagttgatactgtggcagtaaactattaaaagttttcactgttcaaaaaaaaaaaaaaaaaa

SEQ ID NO: 14 - DNA, 1516 bp, Homo sapiens
tgtaagggcacatcccgcgagctgccgcccagcgcgcagacagagcccaggggagcaagagaacgggcgggcggtggggctcacggcctagggaggcgcggaggcatctggcagaggcgggtcgggctgggccagctggggtagagcggaggagcgggtgccggctgaagcggggcggtgggcgcggagcgcgctgggggcaccgacaccacctcaccggcagccgggtgctgagggccgcggtgtgggtgcgcggagcagtcagggcgcaggtgggcagcgcgcacggcctgccagcccggggcgccagaatcctgcgctgcggggccgagaggggcgccgcgcccgccgcagcctggagctttccgcgaacctcggggcgcccatgacggcggcggcgacggctaccgtgctcaaggagggcgtgctggagaagcgcagcggcgggctgctgcagctgtggaagcggaagcgctgcgtcctcaccgaacgcgggctgcagctcttcgaggccaagggcacgggcggccggcccaaggagctcagcttcgcccgcatcaaggccgtggagtgcgtggagagcaccgggcgccacatctacttcacgctggtgaccgaagggggcggcgagatcgacttccgctgccccctggaagatcccggctggaacgcccagatcaccctaggcctggtcaagttcaagaaccagcaggccatccagacagtgcgggcccggcagagcctcgggaccgggaccctcgtgtcctaaaccaccgggcgcaccatctttccttcatgctacccaccacctcagtgctgaggtcaaggcagcttcgttgttccctctggcttgtgggggcacggctgtgctccatgtggcaaggtggaaggcatggacgtgtggaggaggcgctggagctgaaggaatggacgagccctgggaggagggcagaaggctacgcagggctgaggatgaagatgcagcccctggatggtcccagactctcaggacatgcccagctcaggggcttcgagccacaggcctggcctcatatggcatgagggggagctggcataggagccccctccctgctgtggtcctgccctctgtcctgcagactgctcttagccccctggctttgtgccaggcctggaggagggcagtcccccatggggtgccgagccaacgcctcaggaatcaggaggccagcctggtaccaaaaggagtacccagggcctggtacccaggcccactccagaatggcctctggactcaccttgagaagggggagctgctgggcctaaagcccactcctgggggtctcctgctgcttaggtccttttgggacccccacccatccaggccctttctttgcacacttcttcccccacctctacgcatcttccccccactgcggtgttcggcctgaaggtggtgggggtgagggggggtttggccattagcatttcatgtctttccccaaatgaagatgccctgcaaagggcagtaaccaca

SEQ ID NO: 15 - DNA, 7495 bp, Homo sapiens
gtagggggcgcgcaccgaggcaccgcggcgagcttggctgcttctggggcctgtgtggccctgtgtgtcggaaagatggagcaagaagccgagcccgaggggcggccgcgacccctctgaccgagatcctgctgctttcgcagccaggagcaccgtccctccccggattagtgcgtacgagcgcccagtgccctggcccggagagtggaatgatccccgaggcccagggcgtcgtgcttccgcgcgccccgtgaaggaaactggggagtcttgagggacccccgactccaagcgcgaaaaccccggatggtgaggagcaggcaaatgtgcaataccaacatgtctgtacctactgatggtgctgtaaccacctcacagattccagcttcggaacaagagaccctggttagaccaaagccattgcttttgaagttattaaagtctgttggtgcacaaaaagacacttatactatgaaagaggttcttttttatcttggccagtatattatgactaaacgattatatgatgagaagcaacaacatattgtatattgttcaaatgatcttctaggagatttgtttggcgtgccaagcttctctgtgaaagagcacaggaaaatatataccatgatctacaggaacttggtagtagtcaatcagcaggaatcatcggactcaggtacatctgtgagtgagaacaggtgtcaccttgaaggtgggagtgatcaaaaggaccttgtacaagagcttcaggaagagaaaccttcatcttcacatttggtttctagaccatctacctcatctagaaggagagcaattagtgagacagaagaaaattcagatgaattatctggtgaacgacaaagaaaacgccacaaatctgatagtatttccctttcctttgatgaaagcctggctctgtgtgtaataagggagatatgttgtgaaagaagcagtagcagtgaatctacagggacgccatcgaatccggatcttgatgctggtgtaagtgaacattcaggtgattggttggatcaggattcagtttcagatcagtttagtgtagaatttgaagttgaatctctcgactcagaagattatagccttagtgaagaaggacaagaactctcagatgaagatgatgaggtatatcaagttactgtgtatcaggcaggggagagtgatacagattcatttgaagaagatcctgaaatttccttagctgactattggaaatgcacttcatgcaatgaaatgaatcccccccttccatcacattgcaacagatgttgggcccttcgtgagaattggcttcctgaagataaagggaaagataaaggggaaatctctgagaaagccaaactggaaaactcaacacaagctgaagagggctttgatgttcctgattgtaaaaaaactatagtgaatgattccagagagtcatgtgttgaggaaaatgatgataaaattacacaagcttcacaatcacaagaaagtgaagactattctcagccatcaacttctagtagcattatttatagcagccaagaagatgtgaaagagtttgaaagggaagaaacccaagacaaagaagagagtgtggaatctagtttgccccttaatgccattgaaccttgtgtgatttgtcaaggtcgacctaaaaatggttgcattgtccatggcaaaacaggacatcttatggcctgctttacatgtgcaaagaagctaaagaaaaggaataagccctgcccagtatgtagacaaccaattcaaatgattgtgctaacttatttcccctagttgacctgtctataagagaattatatatttctaactatataaccctaggaatttagacaacctgaaatttattcacatatatcaaagtgagaaaatgcctcaattcacatagatttcttctctttagtataattgacctactttggtagtggaatagtgaatacttactataatttgacttgaatatgtagctcatcctttacaccaactcctaattttaaataatttctactctgtcttaaatgagaagtacttggtttttttttttcttaaatatgtatatgacatttaaatgtaacttattattttttttgagaccgagtcttgctctgttacccaggctggagtgcagtggcgtgatcttggctcactgcaagctctgcctcccgggttcgcaccattctcctgcctcagcctcccaattagcttggcctacagtcatctgccaccacacctggctaattttttgtacttttagtagagacagggtttcaccgtgttagccaggatggtctcgatctcctgacctcgtgatccgcccacctcggcctcccaaagtgctgggattacaggcatgagccaccgcgtccggcctaaatgtcacttagtacctttgatataaagagaaaatgtgtgaaagatttagttttttgtttttttgtttgtttgtttgtttgtttgttttgagatgagtctctctgtcgcccaggctggagtgcagtgtcatgatctagcagtctccgcttcccgggttcaagccattctcctggctcagcctctggagcagctgggattacaggcatgcaccaccatgcccagctaatttttgtatttttagtagagatagggtttcaccatgttggccaggctggtcacgaactcctgacctcaagtgaggtcacccgcctcggcctcccgaagtgctgggattgcagatgtgagccaccatgtccagccaagaattagtatttaaattttagatactctttttttttttttttttttttttttttgagacagagtcttgctccatcacccatgctagagtgcagtggagtgatctcggctcactgcaacttccgccttctgggttcaagctattctcctgcctcagccttccaagtaactgggattacaggcatgtaccaccataccagctgatttttttgtatttttagtaaagacagggtttcaccatgttagccaggctgatcttgaactcctaaactcaagtgatctactcacctcagcctcccaaaatgctgggattacagatgtgaggcacctggcctcagatttttgatactcttaaaccttctgatccttagtttctctctccaaaatactctttctaggttaaaaaaaaaaaggctcttatatttggtgctatgtaaatgaaaatgttttttaggttttcttgatttaacaatagagacagggtctccctgtgttgcccaggctggtctcgaactcctgggctcaagagatcctcctgtcttggcctcgcaaagtgctaagtaggattacaggcgttagccaccacacccggctgtaaaaatgtacttattctccagcctcttttgtataaaccatagtaagggatgggagtaatgatgttatctgtgaaaatagccaccatttacccgtaagacaaaacttgttaaagcctcctgagtctaacctagattacatcaggccctttttcacacacaaaaaaatcctttatgggatttaatggaatctgttgtttccccctaagttgaaaaacaactctaagacactttaaagtaccttcttggcctgggttacatggttcccagcctaggtttcagacttttgcttaaggccagttttagaaacccgtgaattcagaaaagttaattcagaaatttgataaacagaattgttatttaaaaactaactggaaagattgttaagttctttctgaattattcagaaattatgcatcattttccttcaagaatgacagggtcagcatgtggaattccaagatacctcttgacttcctctcaagctccgtgtttggtcagtggaggcccatccgagctcagcactgagaagtgttagtttctttgggacccatctaccctgaccacatcatgatgttcatctgcagctgttgcaaggtgttcagattgtataaacataaatgtcacaaaaactttaaaagaagtgcaattctcaaaaggttaggtggactaaagcattctgtaaagcaactgctaataatgagcttacagtggatttgaatttgaaaaatatagtaacaagcctgtcaaatatctgcaagaactatggaataaaactactgatgcagtgaagacagttgaaaagatcaaacaaatgccaagctatatttataatgaacaaattcaagaaaaaggactacggaaagttcaggacatcaaagaagtcaggcaaaactcatcttgacccctgttgcaggcaaaggaacgcagctggaagaaaagatgatataacagttaacaggatgcagacatggcagaggtttcctaaaaatctcattatctataaccatttctatatttacatttgaaaatctcctttggagacttagaacctctaaattattgacttattttttatataaggtcactccgatgaaaggtgattacaaaatcatctacattgctgtctacaaaacagataatatggatgtttgatcgcatctcattgttaactctttactgatatgtttgtaaatacagaagtgaaatgtggacataaaatagttacgctatttggttaatggtactagacaacatgtaattaatgacattcaaaaatttatggctagtgatatatataaagtaaaattttctttgcagtaaaatatgccctttattatagaagggaggatataaggaaccaacagtttgtatgaaaatagctcaaataatatcttttattttgattttaatatttcttattttggtttattagtgtcttagaacaaaatggccttatataatgaagcctagttatgctggactgttttgatctcttttaattgttctgacagatagttggggatgagagccgaataaggtttgcctgaaataactgacactatataatttctgctttggcaaatactaagttctaacttgtcattcctggtagaacaagctttatttttcgagcctagcaatgatctagaagcagatgttatctcagtgccttttgcaatttgttgtgtgggtttttttttttttaaagccacacaataattttggaaaacaatgtatgggtagaacatgtgtctgttaattgcacacaaaaccacttttaatgggtacagagttaaatttgaaggaataagttctagctgaagtattatgaactccaaataatgctttgaggacctccaaaggtaaaagtactaatccctttggccatttattgagagagagagagagagagagtagggtgactatagttaatgtattgaatgttcttgctacaaataaatgatatttgagctgatgggtgtgctaattacactgatttgatcaatacccattgtatgtgaaacagtacatacaccatatttacaattatgtatttaacatttaaaatttctaatataagtatctctcaaactgtggattaacttcttgatttatatttaaatatgaatcttaagcaaaacagtgaaaataaccatcttgatttagtgtttttctcccatatgtgaattgtatatacttaggtgaagacaataaaatcaactgaactgtaagcttagaataggactgaggtaattctgcacagcaactttactaatggtacattgttgcttcaaaactctctctctctctctctgtctgtctcaataaatggccaaagggattagtagtttacctgtggaggtcctccaagcattatttggagttgataatacttcagctacaaccaagcagaatctcttttttttggaggtcctcgaagcattatttggagttgataatacttcagcttcaatttggagttgataatatttcagctagaacctagtagaatctgtttttttcctttggaggtcctcaaagcattattggagttcataatactgaagctagaaccaagcagaatctgtttttttctgaggagtatcggtagcataaatgtgattataaacatagtacacttgatatatggaggcagtgacagctatttttacaaaatttaaatctgcaaatggattcaacatgtttatgggttattaaaattgtctgatttcttaggttctttatagtacacgtgttgaaaataaatgattaagaattgtttcaagaatgcaattatttgatcttaaatttttatgagttgttaaaatagaaattatttgaatatcatatatttgggtaacaaaaggcacaagtctgaatgtgtttctttttctggaatggccatgcctgcccactttagaaatacaaatatcactgggcagcttgaagcagttgggagcctccaatgagagcaacttgagagaatgatgttgcaagttagtaggagtaagaaatgctgtgttctccctgtcttctcttaggtcacatggcagcctggcctaagtgatcgtgaatggtctataagggaggtagctgggacagggaggggagtttgggctagccaccgtaccacttgtcagcgtgaaaagtaagattgtaattgcctgtttagttttctgcctcatctttgaaagttccaccaagctgggaacctcttgattgtgaggcacaaatgtaagtacatcagaaaaaaacaaaaaaactggctttaaagcaggagcttgtgggcccctaagccagacggggactagcttttggcattatataattaagattttttaaatccttaataagggttttattttatttttatttattttttgagacggagtcttgctctgtggctcaggctggagtacagtggtgcaatcttggctcactgcaacctctgcctcctggctgtgttcaagtggttctgcttcagcctcccaagtagctggggttagagcaccctgtcaccacgccccgctaatttttgtatttctagcagagatgaagtttcactatgttggccaggctgggctcaaactcctgacctcaagtgatctgcccgccttggccccccaaagtgctgtgattacaggcgtgagccgccacgcccagcctaataagggttttaaagataattagtgtgtaggtctgtaggcttatgatggtaaccacaagttgttaatggcattgtgaaaagtttttagttgcgctttatgggtggatgctgaattacattttgatttgatacttataaaaagaaaaagtatttcttcagcttaaaaaattgtttaaaagtttgtgatcatattgtctaccatgtagccagctttcaattatatgtaagagggactttttgacatttacaaataatactttgaggtagatatctgaaagcaccagcacttggaaggtgttcagaagtaacaaattataaaatgagctaacaaacgaaaggcaaaataaaaccgtaaagcaagcagatgggaggcgtgttcagtaacttattcataatgcatctgaaatgattgctgtactcaaatatttaacgttagagtaatagtattttgaatgaaaaccatagttgattgtct

SEQ ID NO: 16 - DNA, 2091 bp, Homo sapiens
gagcggagccgcgggcgggagggcggacggaccgactgacggtagggacgggaggcgagcaagatggcgcagacgcagggcacccggaggaaagtctgttactactacgacggggatgttggaaattactattatggacaaggccacccaatgaagcctcaccgaatccgcatgactcataatttgctgctcaactatggtctctaccgaaaaatggaaatctatcgccctcacaaagccaatgctgaggagatgaccaagtaccacagcgatgactacattaaattcttgcgctccatccgtccagataacatgtcggagtacagcaagcagatgcagagattcaacgttggtgaggactgtccagtattcgatggcctgtttgagttctgtcagttgtctactggtggttctgtggcaagtgctgtgaaacttaataagcagcagacggacatcgctgtgaattgggctgggggcctgcaccatgcaaagaagtccgaggcatctggcttctgttacgtcaatgatatcgtcttggccatcctggaactgctaaagtatcaccagagggtgctgtacattgacattgatattcaccatggtgacggcgtggaagaggccttctacaccacggaccgggtcatgactgtgtcctttcataagtatggagagtacttcccaggaactggggacctacgggatatcggggctggcaaaggcaagtattatgctgttaactacccgctccgagacgggattgatgacgagtcctatgaggccattttcaagccggtcatgtccaaagtaatggagatgttccagcctagtgcggtggtcttacagtgtggctcagactccctatctggggatcggttaggttgcttcaatctaactatcaaaggacacgccaagtgtgtggaatttgtcaagagctttaacctgcctatgctgatgctgggaggcggtggttacaccattcgtaacgttgcccggtgctggacatatgagacagctgtggccctggatacggagatccctaatgagcttccatacaatgactactttgaatactttggaccagatttcaagctccacatcagtccttccaatatgactaaccagaacacgaatgagtacctggagaagatcaaacagcgactgtttgagaaccttagaatgctgccgcacgcacctggggtccaaatgcaggcgattcctgaggacgccatccctgaggagagtggcgatgaggacgaagacgaccctgacaagcgcatctcgatctgctcctctgacaaacgaattgcctgtgaggaagagttctccgattctgaagaggagggagaggggggccgcaagaactcttccaacttcaaaaaagccaagagagtcaaaacagaggatgaaaaagagaaagacccagaggagaagaaagaagtcaccgaagaggagaaaaccaaggaggagaagccagaagccaaaggggtcaaggaggaggtcaagttggcctgaatggacctctccagctctggcttcctgctgagtccctcacgtttcttccccaacccctcagattttatattttctatttctctgtgtatttatataaaaatttattaaatataaatatccccagggacagaaaccaaggccccgagctcagggcagctgtgctgggtgagctcttccaggagccaccttgccacccattcttcccgttcttaactttgaaccataaagggtgccaggtctgggtgaaagggatacttttatgcaaccataagacaaactcctgaaatgccaagtgcctgcttagtagctttggaaaggtgcccttattgaacattctagaaggggtggctgggtcttcaaggatctcctgtttttttcaggctcctaaagtaacatcagccatttttagattggttctgttttcgtaccttcccactggcctcaagtgagccaagaaacactgcctgccctctgtctgtcttctcctaattctgcaggtggaggttgctagtctagtttcctttttgagatactattttcatttttgtgagcctctttgtaataaaatggtacatttct

SEQ ID NO: 17 - DNA, 41 bp, Artificial Sequence
  Synthetic primer
cctcaagtgagccaagaaacaataactgccctctgtctgtc

SEQ ID NO: 18 - DNA, 41 bp, Artificial Sequence
  Synthetic primer
gacagacagagggcagttattgtttcttggctcacttgagg

SEQ ID NO: 19 - DNA, 43 bp, Artificial Sequence
  Synthetic primer
ggcctcaagtgagccaaaaataaataactgccctctgtctgtc

SEQ ID NO: 20 - DNA, 43 bp, Artificial Sequence
  Synthetic primer
gacagacagagggcagttatttatttttggctcacttgaggcc

SEQ ID NO: 21 - RNA, 48 nt, Homo sapiens
uaccuucccacuggccucaagugagccaaaagacacugccugcccucu

SEQ ID NO: 22 - RNA, 48 nt, Homo sapiens
uaccuucccacuggccucaagugagccaaaaauaaauaacugcccucu

SEQ ID NO: 23 - RNA, 18 nt, Artificial Sequence
  Description of Artificial Sequence Synthetic oligonucleotide
ggcaguguuuagcuguug